(19) United States
(12) Patent Application Publication
(10) Pub. No.: US 2026/0233000 A1
(43) Pub. Date: Aug. 13, 2026

(54) Methods, Systems, and Apparatuses, for Managing Gait Operation in a Neuroprosthesis

(71) Applicants: The United States Government as represented by the Department of Veterans Affairs, Washington, DC (US); Case Western Reserve University, Cleveland, OH (US)

(72) Inventors: Ryan-David Reyes, Tallahassee, FL (US); Roger D. Quinn, Akron, OH (US); Ronald J. Triolo, Cleveland Heights, OH (US); Musa L. Audu, Shaker Heights, OH (US); Nathaniel S. Makowski, Cleveland, OH (US)

(21) Appl. No.: 19/154,698

(22) PCT Filed: Feb. 12, 2024
(86) PCT No.: PCT/US2024/015364

§ 371 Date: Aug. 7, 2025

Related U.S. Application Data
(60) Provisional application No. 63/444,825, filed on Feb. 10, 2023.

(51) Int. Cl. A61N 1/36; A61B 5/11; A61H 3/00

(52) U.S. Cl. CPC A61N 1/36003; A61B 5/112; A61B 5/1121; A61H 3/00; A61H 2201/1215; A61H 2201/1642

(57) ABSTRACT

Methods, systems, and/or apparatuses are described for providing electrical stimuli and motorized assistance for leg movement of a user. A quantity of electrical stimuli may be provided to a portion of a paretic leg during the first step of a walking motion. Motorized assistance at a quantity of torque may be provided to the portion of the paretic leg during the first step of the walking motion. Data associated with the paretic leg during the first step of the walking motion may be received. Based on the data, the deviation of the paretic leg, during the first step of the walking motion, from the target configuration for the paretic leg may be determined. The quantity of electrical stimuli during the next step of the walking motion for the paretic leg may be modified.

### CROSS-REFERENCE TO RELATED APPLICATIONS

This application claims priority to and the benefit of U.S. Provisional Patent Application No. 63/444,825, filed Feb. 10, 2023, the entire contents of which are hereby incorporated herein by reference in its entirety.

### BACKGROUND

Stroke survivors, as well as those with other ailments, may suffer from partial paralysis or reduced capabilities in one or both of their legs (e.g., one or both paretic legs). These people may have difficulty walking or relearning to walk without some sort of assistance. Providing motorized assistance or electrical stimulation to portions of the paretic leg through different portions of the gait cycle has the potential to substantially improve walking. Conventional devices such as hybrid exoskeletons combine motor assistance and electrical stimulation to integrate the biological motive power generated by the muscles with the torques generated by the motorized bracing to accomplish ambulatory motion. However, certain characteristics of this combined system make the coordination difficult. For example, muscle contractions elicited by electrical stimulation represent highly nonlinear time-varying systems. In contrast, the motorized assistance can be modeled as linear time-invariant systems. Thus, the conventional devices exhibit actuator redundancy with multiple muscles and a motor acting on the same joint. Furthermore, conventional devices do not intelligently allocate control effort between muscles and motors.

### SUMMARY

Described herein, in various aspects, are methods, systems, and apparatuses configured to provide electrical stimuli and motorized assistance for walking. For example, the control of electrical stimuli and motorized assistance may be provided by an apparatus that is removably coupled to one or both legs (and optionally at least a portion of the torso) of a person (e.g., a survivor or user). For example, one or both legs of the user may be paretic legs caused by a previously suffered a stroke or other ailment.

In certain examples, the apparatus may comprise a neuroprosthesis, exoskeleton, or brace (hereinafter referred to as a brace). The brace may comprise a thigh section, a shank section, and a foot support section. The thigh section and the shank section may be movably coupled to one another and the shank section and the foot support section may be movably coupled to one another. The brace may comprise a motor configured to provide motorized assistance between the thigh section and the shank section. The brace may comprise one or more electrodes and an electrical power source electrically coupled thereto. The one or more electrodes may be configured to provide electrical stimuli to a thigh portion and/or shank portion of the paretic leg of the user. The brace may comprise one or more sensors configured to determine kinematic information associated with all or a particular portion of the paretic leg. For example, the brace may comprise one or more of a thigh sensor, a shank sensor, or a heel strike sensor. For example, heel strike sensors may be provided for both the foot of the paretic leg and the foot of the non-paretic leg of the user. The heel strike sensors may also be provided for both foots of the paretic legs of the user. The brace may comprise one or more devices or mechanisms for attaching the brace to the paretic leg of the user. The apparatus may comprise a control computing device. The control computing device may be a computer configured to receive sensor data from the one or more sensors and determine to initiate, terminate, increase, decrease, or modify one or more of the motorized assistance and/or the electrical stimuli to all or a portion of the paretic leg(s).

In certain examples, a method for providing electrical stimuli and motorized assistance for walking may be provided. For example, a first quantity of electrical stimuli may be provided to a paretic leg during the first step of a walking motion. Motorized assistance at a first quantity of torque may be provided for the paretic leg during the first step of the walking motion. Data associated with the paretic leg during the first step of the walking motion may be received. A deviation of the paretic leg from a target configuration may be determined. The deviation may be determined during the first step of the walking motion. The first quantity of electrical stimuli during the next step of the walking motion for the paretic leg may be modified based on the deviation. Alternatively or additionally, the first quantity of torque provided by the motorized assistance during a next step of the walking motion for the paretic leg may be modified based on the deviation.

In certain examples, a method for providing electrical stimuli and motorized assistance for walking may be provided. For example, a first quantity of electrical stimuli may be provided to a portion of a paretic leg during a first step of a walking motion. Motorized assistance may be provided for the paretic leg at a first quantity of torque during the first step of the walking motion. Data associated with the paretic leg during the first step of the walking motion may be received. Based on the received data, a movement of the paretic leg may be determined. The movement of the paretic leg may be determined during the first step of the walking motion. The movement of the paretic leg may satisfy a target configuration for the paretic leg during the first step of the walking motion. Based on the movement satisfying the target configuration, the first quantity of torque provided by the motorized assistance may be reduced during a second step of the walking motion for the paretic leg.

This summary is not intended to identify critical or essential features of the disclosure, but merely to summarize certain features and variations thereof. Other details and features will be described in the sections that follow. Additional advantages of the disclosure will be set forth in part in the description that follows, and in part will be obvious from the description, or may be learned by practice of the concepts described in this disclosure. The advantages of the concepts described in this disclosure will be realized and attained by means of the elements and combinations particularly pointed out in the appended claims. It is to be understood that both the foregoing general description and the following detailed description are exemplary and explanatory only and do not restrict the scope of the claims.

### DESCRIPTION OF THE DRAWINGS

The accompanying drawings, which are incorporated in and constitute a part of the present description serve to explain the principles of the methods, apparatuses, and systems described herein:

FIG. 1 shows an example system for providing mechanical and/or electrical assistance for leg movement;

FIG. 2 shows an example system for providing electrical assistance for leg movement;

FIG. 3A shows an example system for providing mechanical and/or electrical assistance for paretic or partially paretic leg movement;

FIG. 3B shows an example system for providing electrical assistance for paretic or partially paretic leg movement;

FIG. 4 shows an example graph for hip and knee errors per iteration;

FIG. 5A shows an example graph for hip angle over time per iteration;

FIG. 5B shows an example graph for knee angle over time per iteration;

FIG. 6 shows an example graph for hip and knee burst torques per iteration;

FIG. 7 shows an example graph for hip and knee errors per iteration;

FIG. 8 shows an example graph for hip and knee burst torques per iteration;

FIG. 9 shows an example method for providing electrical and/or mechanical assistance for leg movement;

FIG. 10 shows another example method for providing electrical and/or mechanical assistance for leg movement;

FIG. 11 shows another example method for providing electrical and/or mechanical assistance for leg movement;

FIG. 12 shows another example method for providing electrical and/or mechanical assistance for leg movement;

FIG. 13 shows an example system for machine-learning to provide electrical and/or mechanical assistance for leg movement;

FIG. 14 shows an example method for machine-learning to provide electrical and/or mechanical assistance for leg movement; and

FIG. 15 shows example system for providing mechanical and/or electrical assistance for leg movement.

### DETAILED DESCRIPTION

Before the present methods, systems, and apparatuses are disclosed and described, it is to be understood that the methods, systems, and apparatuses are not limited to specific methods, specific components, or to particular implementations. It is also to be understood that the terminology used herein is for the purpose of describing particular embodiments only, and is not intended to be limiting.

As used in the specification and the appended claims, the singular forms “a,” “an” and “the” include plural references unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Furthermore, descriptions of an event or circumstance without use of “optional” or “optionally” does not mean that the described event does occur, must occur, or is necessary to the operation of the apparatus or system or required for the performance of the method.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods, apparatuses, and systems. This applies to all aspects of this application including, but not limited to, steps in disclosed methods. Thus, if there are a variety of additional steps that may be performed it is understood that each of these additional steps may be performed with any specific embodiment or combination of embodiments of the disclosed methods.

The present methods, systems, and apparatuses may be understood more readily by reference to the following detailed description of example embodiments and the examples included therein and to the figures and their previous and following description.

As will be appreciated by one skilled in the art, one or more of the methods, systems, and apparatuses described herein may take the form of an entirely hardware embodiment, an entirely software embodiment, or an embodiment combining software and hardware aspects. Furthermore, the methods, systems, and apparatuses may take the form of a computer program product on a computer-readable storage medium (e.g., a non-transitory computer-readable medium) and having computer-readable program instructions (e.g., computer software) embodied in the storage medium. Any suitable computer-readable storage medium (e.g., a non-transitory computer-readable medium) may be utilized including hard disks, CD-ROMs, optical storage devices, flash drive, SD card or similar non-volatile memory card, or magnetic storage devices.

Embodiments of the methods, systems, and apparatuses are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a microcontroller, general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce functions on an article of manufacture including computer-readable instructions for implementing the function specified in the flowchart block or blocks. The computer program instructions may also be loaded onto a microcontroller, computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems or one or more microcontrollers that perform the specified functions or steps, or combinations of special purpose hardware and computer instructions.

Hereinafter, various embodiments of the present disclosure will be described with reference to the accompanying drawings. As used herein, the term “user” may indicate a person.

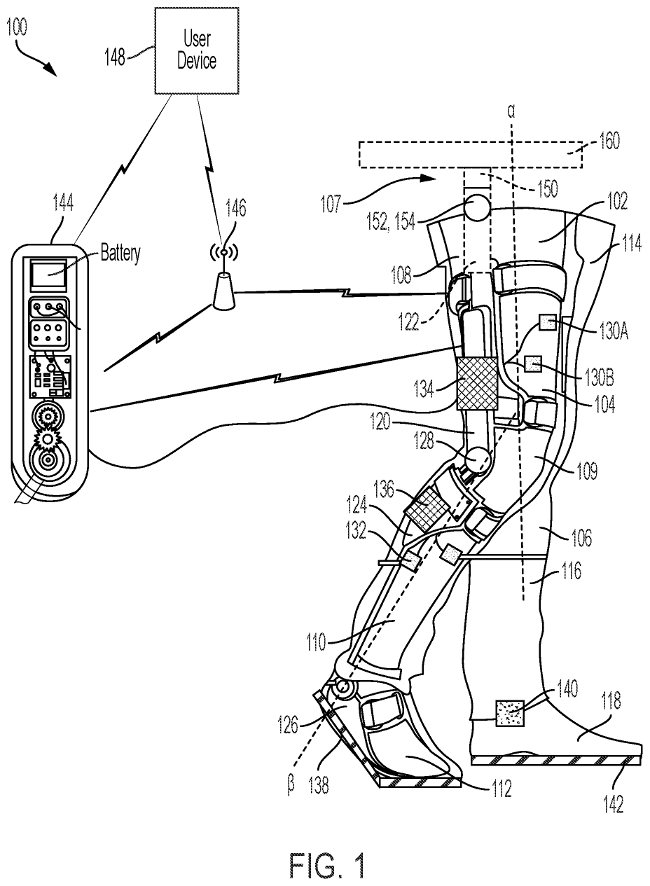

FIG. 1 shows an example system 100 for providing mechanical and/or electrical assistance for leg movement. For example, the assistance may be provided to a user 102, such as a person. The user 102 may have a paretic leg 104 (e.g., a leg suffering partial paralysis) and a non-paretic leg 106. While the example of FIG. 1 shows the right leg of the user 102 being the paretic leg 104, this is for example purposes only, as the systems and methods described herein would equally work if the left leg of the user 102 was the paretic leg 104. In certain examples, both legs of the user 102 may be paretic legs (not shown). The paretic leg 104 or both paretic legs may have been caused by a stroke or other ailment or injury suffered by the user 102. Although it is not shown in FIG. 1, the system 100 applied to the paretic leg 104 may also apply to the other paretic leg (e.g., in case both legs of the user 102 are paretic).

The paretic leg 104 may comprise a pelvic portion 107, a thigh portion 108, a knee portion 109, a shank portion 110, and a foot 112. For example, the thigh portion 108 may be the portion of the paretic leg 104 between the pelvic portion 107 (e.g., the pelvis) and the knee portion 109 of the paretic leg 104. For example, the shank portion 110 may be the portion of the paretic leg 104 between the knee portion 109 and the foot 112 of the paretic leg 104. The knee portion 109 may be the portion of the paretic leg 104 providing an axis of rotation for the shank portion 110 with respect to the thigh portion 108. A hip may be the portion of the paretic leg 104 providing an axis of rotation for the thigh portion 108 with respect to the pelvic portion 107, torso, or trunk of the user 102.

The non-paretic leg 106 or the other paretic leg (not shown) may comprise a thigh portion 114, a shank portion 116, and a foot 118. For example, the thigh portion 114 may be the portion of the non-paretic leg 106 or the other paretic leg (not shown) between the pelvis and the knee of the non-paretic leg 106 or the other paretic leg (now shown). For example, the shank portion 116 may be the portion of the non-paretic leg 106 or the other paretic let (not shown) between the knee and the foot 118 of the non-paretic leg 106 or the other paretic leg (not shown).

The system 100 may comprise a neuroprosthesis, exoskeleton, hybrid exoskeleton, or brace 120 (referred to hereinafter as the brace 120). For example, the brace 120 may be a leg brace. For example, the brace 120 may be configured to be attached to one (e.g., the paretic leg 104) or both paretic legs of the user 102. The brace 120 may be made of one or more of plastic or metal components. For example, the brace 120 may comprise one or more straps, belts, or the like for removably attaching the brace 120 to the paretic leg 104 or another portion (e.g., the waist) of the user 102. For example, the one or more straps may comprise hook and loop straps that provide adjustability in attaching the brace 120 to the paretic leg 104 or other portion of the user 102.

The brace 120 may comprise one or more sections. For example, the brace 120 may comprise a thigh section 122, a shank section 124, and a foot support section 126. In certain examples, the brace 120 may also comprise a hip section 150, and a waist section 160 for attaching the brace 120 around the user's waist. The thigh section 122 may be movably coupled to the shank section 124 and may be configured to move or rotate with respect to the shank section 124. In certain examples, the thigh section 122 may also be movably coupled to the hip section 150 and may be configured to move or rotate with respect to the hip section 150. The thigh section 122 may include an elongated support member. The elongated support member may be configured to extend along at least a portion of the thigh portion (e.g., upper leg) 108 of the user 102. For example, the thigh section 122 may be configured to be positioned along an outer side of the thigh portion 108 of the paretic leg 104. The thigh section 122 may comprise one or more straps, belts, or the like for removably attaching the thigh section 122 to the thigh portion 108 of the paretic leg 104. For example, the one or more straps may comprise hook and loop straps that provide adjustability in attaching the thigh section 122 to the thigh portion 108.

The shank section 124 may be movably coupled to the thigh section 122 and may be configured to move or rotate with respect to the thigh section 122. The shank section 124 may be movably coupled to the foot support section 126 and may be configured to move or rotate with respect to the foot support section 126. The shank section 122 may include an elongated support member. The elongated support member may be configured to extend along at least a portion of the shank portion (e.g., lower leg) 110 of the paretic leg 104. For example, the shank section 124 may be configured to be positioned along an outer side and/or back side of the shank portion 110 of the paretic leg 104. The shank section 124 may comprise one or more straps, belts, or the like for removably attaching the shank section 124 to the shank portion 110 of the paretic leg 104. For example, the one or more straps may comprise hook and loop straps that provide adjustability in attaching the shank section 124 to the shank portion 110 of the paretic leg 104.

The foot support section 126 may be movably coupled to the shank section 124 and may be configured to move or rotate with respect to the shank section 124. The foot support section 126 may include one or more panels. The one or more panels may comprise a bottom panel configured to contact a bottom side of the foot 112 of the paretic leg 104. The one or more panels may also comprise one or more side panels or a rear panel extending up from the bottom panel and configured to be positioned along an outer perimeter of the foot 112. The foot support section 126 may comprise one or more straps, belts, or the like for removably attaching the foot support section 126 to the foot 112 of the user 102. For example, the one or more straps may comprise hook and loop straps that provide adjustability in attaching the foot support section 126 to the foot 112.

The hip section 150 may be movably coupled to the thigh section 122 and may be configured to move or rotate with respect to the thigh section 122. The hip section 150 may extend from the thigh section 122 to the waist section 160. The hip section 150 may include a support member (e.g., an elongated support member). The support member may be configured to extend along at least a portion of the pelvic portion 107 of the paretic leg 104. For example, the hip section 150 may be configured to be positioned along an outer side of the pelvic portion 107 of the paretic leg 104.

The waist section 160 may be coupled to the hip section 150. The waist section 160 may be configured to extend around the waist or trunk/torso of the user 102. The waist section 160 may comprise one or more straps, belts, or the like for removably attaching the waist section 160 around the waist/torso/trunk of the user 102. For example, the one or more straps may comprise hook and loop straps that provide adjustability in attaching the waist section 160 to the waist/torso/trunk of the user 102.

The brace 120 may comprise one or more motors 128. The one or more motors 128 may be positioned at or near an axis of rotation between the thigh section 122 and the shank section 124. In certain examples, another one or more motors 152 may be positioned at or near an axis of rotation between the thigh section 122 and the hip section 150. For example, the one or more motors 152 may be provided for users 102 that have limited active hip motion. In certain examples, additional motors may be provided, such as a motor between the shank section 124 and the foot support section 126 to control rotation of the foot 112 with respect to the shank section 110 of the paretic leg 104. The one or more motors 128 may be configured to provide motorized assistance with respect to the shank portion 110 rotating with respect to the thigh portion 108 of the paretic leg 104 by providing motorized assistance for the shank section 124 to rotate with respect to the thigh section 122 of the brace 120. In other examples, the one or more motors 128 may be configured to provide motorized resistance with respect to the shank portion 110 rotating with respect to the thigh portion 108 by providing motorized resistance against the shank section 124 rotating with respect to the thigh section 122 of the brace 120. The one or more motors 152 may be configured to provide motorized assistance with respect to the thigh portion 108 rotating with respect to the pelvic portion 107 of the paretic leg 104 by providing motorized assistance for the thigh section 122 to rotate with respect to the hip section 150 of the brace 120. While one example of providing motorized assistance for the thigh portion 108 with respect to the pelvic portion 107, other examples are possible. For example, cabling could be attached to textiles worn on the leg of the user 102 to generate the torques for mobilizing the thing portion 108 with respect to the pelvic portion 107. For example, the one or more motors 152 may provide motorized assistance with hip flexion at the end of the terminal stance phase and then during early, mid, and terminal swing. Motorized assistance may reduce during terminal swing and the one or more motors 152 may provide motorized assistance with hip/thigh extension from heel strike to midstance. In other examples, the one or more motors 152 may be configured to provide motorized resistance with respect to the thigh portion 108 rotating with respect to the pelvic portion 107 by providing motorized resistance against the thigh section 122 rotating with respect to the hip section 150 of the brace 120. The motorized resistance may be provided in order to help build muscle strength in one or more portions of the paretic leg 104.

The one or more motors 128 may include or be operably coupled to a sensor 129. For example, the sensor 129 may be an encoder. The sensor 129 may provide rotational data indicating the amount of rotation of the shank portion 110 with respect to the thigh portion 108 of the paretic leg 104. The one or more motors 128 and the sensor 129 may be electrically coupled to a power source (not shown). The one or more motors 152 may include or be operably coupled to a sensor 154. For example, the sensor 154 may be an encoder. The sensor 154 may provide rotational data indicating the amount of rotation of the thigh portion 108 with respect to the pelvic portion 107 of the paretic leg 104. The one or more motors 152 and the sensor 154 may be electrically coupled to a power source (not shown). The power source may be coupled to the brace 120 and may be configured to provide electrical power to one or more components of the brace 120. For example, the power source may be a battery, battery pack, or backpack battery, such as a rechargeable battery. For example, the power source may be one or more of a lead-acid rechargeable battery, a nickel-cadmium rechargeable battery, a nickel-metal hydride rechargeable battery, or a lithium-ion rechargeable battery.

The brace 120 may comprise an ankle sensor 156. For example, the ankle sensor 156 may be an encoder. The ankle sensor 156 may provide rotational data indicating the amount of rotation of the foot 112 with respect to the shank portion 110 of the paretic leg 104. The ankle sensor 156 may be electrically coupled to the power source for the brace 120.

The brace 120 may comprise one or more electrodes 130A-B, 132. The one or more electrodes 130A-B may be positioned at one or more locations along the outer surface of the thigh portion 108 of the paretic leg 104. The one or more electrodes 132 may be positioned at one or more locations along the outer surface of the shank portion 110 of the paretic leg 104. Additional electrodes (not shown) may be positioned along these and/or other portions of the paretic leg, such as along the pelvic portion 107 and/or the knee portion 109. The one or more electrodes 130A-B, 132 may be configured to provide electrical stimuli to the muscles of the thigh portion 108 and/or shank portion 110 and/or any other portion or portions of the paretic leg 104 in order to provide assistance with rotation and/or movement of the thigh portion 108 and/or shank portion 110 of the leg 104 during movement. The one or more electrodes 130A-B, 132 may be operably coupled to one or more of the sensors 129, 154, 156. The sensor 129 may provide rotational data indicating the amount of rotation of the shank section 124 with respect to the thigh section 122. The sensor 154 may provide rotational data indicating the amount of rotation of the thigh section 122 with respect to the hip section 150. The one or more electrodes 130A-B, 132 may be electrically coupled to the power source.

The brace 120 may comprise a thigh sensor 134. The thigh sensor 134 may be positioned along a portion of the thigh section 122 of the brace 120. For example, the thigh sensor 134 may be coupled to the elongated member of the thigh section 122. For example, the thigh sensor 134 may be an inertial measurement unit or another form of sensor. For example, the thigh sensor 134 may comprise multiple sensors for detecting certain data related to the thigh portion 108 of the paretic leg 104. For example, the thigh sensor 134 may generate or collect data related to the thigh portion 108, the data comprising one or more of acceleration data indicating an acceleration for the thigh portion 108, velocity data (e.g., angular velocity data) indicating a velocity or angular velocity for the thigh portion 108, orientation data indicating an orientation of the thigh portion 108, and/or position data indicating a position of the thigh portion 108 (e.g., position of the thigh portion 108 with respect to the hip or pelvis of the paretic leg 104 or the position of the thigh portion 108 with respect to the shank portion 110 of the paretic leg 104). For example, the thigh sensor 134 may collect data related to the muscle activity along the thigh portion 108 of the paretic leg 104. The muscle activity data may indicate a muscle activity level for the thigh portion 108. The muscle activity level may be compared to a muscle activity threshold. If the muscle activity level satisfies (e.g., is greater than or greater than or equal to) the muscle activity threshold the muscle activity level may indicate an initiation of a phase of the gate cycle and/or a transition from one phase to another phase of the gait cycle.

For example, the orientation data may indicate an angle of orientation of the thigh portion 108 as taken along an elongated axis (a) of the thigh portion 108 as compared to a vertical axis or a horizontal axis. The thigh sensor 134 may be electrically coupled to the power source. The thigh sensor 134 may be communicably coupled to the control computing device 144 and may send one or more of the acceleration data, velocity data (e.g., angular velocity data), orientation data, muscle activity data, and/or position data to the control computing device 144 and/or the user device 148.

The brace 120 may comprise a shank sensor 136. The shank sensor 136 may be positioned along a portion of the shank section 124 of the brace 120. For example, the shank sensor 136 may be coupled to the elongated member of the shank section 124. For example, the shank sensor 136 may be an inertial measurement unit or another form of sensor. For example, the shank sensor 136 may comprise multiple sensors for detecting certain data related to the shank portion 110 of the paretic leg 104. For example, the shank sensor 136 may generate or collect data related to the shank portion 110, the data comprising one or more of acceleration data indicating an acceleration for the shank portion 110, velocity data (e.g., angular velocity data) indicating a velocity or angular velocity for the shank portion 110, orientation data indicating an orientation of the shank portion 110, and/or position data indicating a position of the shank portion 110 (e.g., position of the shank portion 110 with respect to the thigh portion 108 of the paretic leg 104 or the position of the shank portion 110 with respect to the foot 112 of the paretic leg 104). For example, the shank sensor 136 may collect data related to the muscle activity along the shank portion 110 of the paretic leg 104. The muscle activity data may indicate a muscle activity level for the shank portion 108. The muscle activity level may be compared to a second muscle activity threshold. If the muscle activity level satisfies (e.g., is greater than or greater than or equal to) the second muscle activity threshold the muscle activity level may indicate an initiation of a phase of the gate cycle and/or a transition from one phase to another phase of the gait cycle.

For example, the orientation data may indicate an angle of orientation of the shank portion 110 as taken along an elongated axis (B) of the shank portion 110 as compared to a vertical axis or a horizontal axis. The shank sensor 136 may be electrically coupled to the power source. The shank sensor 136 may be communicably coupled to the control computing device 144 and/or the user device 148 and may send one or more of the acceleration data, velocity data (e.g., angular velocity data), orientation data, muscle activity data, and/or position data to the control computing device 144 and/or the user device 148.

The brace 120 may comprise a heel-strike sensor 138. The heel-strike sensor 138 may be positioned along a portion of the foot support section 126 of the brace 120 or along any other portion of the paretic leg 104. For example, the heel-strike sensor may be included as part of one of the other sensors 134, 136. For example, the heel-strike sensor 138 may be coupled to the bottom end or bottom surface of the foot support section 126. For example, the heel-strike sensor 138 may be an inertial measurement unit, a contact sensor, a pressure sensor, or another form of sensor. For example, the heel-strike sensor 138 may indicate when the foot support section 126, the heel of the foot 112 or another portion of the foot 112 of the paretic leg 104 contacts a floor surface. The heel-strike sensor 138 may be electrically coupled to the power source. The heel-strike sensor 138 may be communicably coupled to the control computing device 144 and/or the user device 148 and may send the data indicating the contact by the foot 112 or foot support section 126 with the floor surface to the control computing device 144 and/or the user device 148.

The brace 120 may comprise a foot sensor 140. The foot sensor 140 may be positioned along a portion of the foot 118, ankle, shank section 116, or any other portion of the non-paretic leg 106. For example, the foot sensor 140 may be an inertial measurement unit or another form of sensor. For example, the foot sensor 140 may comprise multiple sensors for detecting certain data related to the non-paretic leg 106. For example, the foot sensor 140 may generate or collect data related to the foot 118, shank portion 116, or another portion of the non-paretic leg 106, the data comprising one or more of acceleration data indicating an acceleration for the foot 118 or shank portion 116, velocity data (e.g., angular velocity data) indicating a velocity or angular velocity for the foot 118 or shank portion 116 (or another portion), orientation data indicating an orientation of the foot 118 or shank portion 116, heel-strike or contact information for the foot 118 along the floor surface and/or position data indicating a position of the foot 118 or shank portion 116. The foot sensor 140 may be electrically coupled to the power source. The foot sensor 140 may be communicably coupled to the control computing device 144 and/or the user device 148 and may send one or more of the acceleration data, velocity data (e.g., angular velocity data), orientation data, heel-strike or contact data, and/or position data to the control computing device 144 and/or the user device 148. Additional sensors (not shown) may be positioned along other portions of the non-paretic leg 106 similar to those described with regard to the paretic leg 104.

The brace 120 may comprise a hip or pelvic (“hip”) sensor. The hip sensor may be positioned along the hip or pelvic region of the user 102. For example, the hip sensor may comprise multiple sensors for detecting certain data related to the hip or pelvic region of the paretic leg 104. For example, the hip sensor may generate or collect data related to the hip or pelvic region, the data comprising one or more of acceleration data indicating an acceleration for the hip region, velocity data (e.g., angular velocity data) indicating a velocity or angular velocity for the hip region, orientation data indicating an orientation of the hip region, and/or position data indicating a position of the hip region. For example, the hip sensor may collect data related to the muscle activity along the hip or pelvic region of the paretic leg 104. The muscle activity data may indicate a muscle activity level for the hip or pelvic region. The muscle activity level may be compared to a third muscle activity threshold. If the muscle activity level satisfies (e.g., is greater than or greater than or equal to) the third muscle activity threshold the muscle activity level may indicate an initiation of a phase of the gate cycle and/or a transition from one phase to another phase of the gait cycle.

The system 100 may comprise a heel-strike sensor 142. The heel-strike sensor 142 may be positioned along a portion of a shoe or foot covering of the foot 118 of the non-paretic leg 106. For example, the heel-strike sensor 142 may be coupled to the bottom end or bottom surface of a shoe. For example, the heel-strike sensor 142 may be an inertial measurement unit, a contact sensor, a pressure sensor, or another form of sensor. For example, the heel-strike sensor 142 may indicate when the heel of the foot 118 or another portion of the foot 118 or shoe of the non-paretic leg 106 contacts the floor surface. The heel-strike sensor 142 may be electrically coupled to the power source. The heel-strike sensor 142 may be communicably coupled to the control computing device 144 and/or the user device 148 and may send the data indicating the contact with the floor surface to the control computing device 144 and/or the user device 148.

The system 100 may comprise a control computing device 144. The control computing device 144 may be a form of computer. The control computing device 144 may be communicably coupled to the sensors 129, 134-142, 154, 156, the motors 128, 152, and/or the electrodes 130A-B, 132. The control computing device 144 may communicate with the sensors 129, 134-142, 154, 156, the motors 128, 152, and/or the electrodes 130A-B, 132 via wired or wireless communication. For example, the control computing device 144 may communicate wirelessly via one or more of Wi-Fi, Bluetooth, Bluetooth Low Energy (BLE), Zigbee, or any other known wireless protocol with the sensors 129, 134-142, 154, 156, the motors 128, 152, and/or the electrodes 130A-B, 132. For example, the control computing device 144 may communicate wirelessly with the brace 120 either directly (e.g., via Bluetooth, BLE, Zigbee, etc.) or via a network (e.g., a WI-FI network), such as via the network device 146 or another network.

The control computing device 144 may comprise one or more processors, one or more memory modules, a power source, a communications module, and/or one or more selection buttons or switches. For example, the one or more processors may comprise any one or more of microcontrollers, microprocessors, or embedded processors. The one or more processers may be configured to receive the data from the one or more sensors 129, 134-142, 154, 156 and determine whether to initiate, terminate, adjust, and/or continue providing one or more of electrical stimuli or motorized assistance at the brace 120. For example, the power source may be a battery, such as a rechargeable battery. For example, the communications module may comprise a transmitter, receiver, or transceiver. The communications module may be configured to receive data from one or more of the sensors 129, 134-142, 154, 156. The communications module may be further configured to send instructions to one or more of the motors 128, 152 and/or the electrodes 130A-B, 132 to provide motorized assistance and/or electrical stimuli to the paretic leg 104.

The system 100 may comprise a user device 148. The user device 148 may be a form of computer (e.g., a computing device). The user device 148 may comprise a desktop computer, a laptop computer, a smart device, a mobile device (e.g., a mobile phone (e.g., a smart phone), a tablet device, a smart watch, etc.), and/or the like. The user device 148 may comprise one or more processors, one or more memory modules, a power source, a communications module, and/or one or more selection buttons or switches. For example, the one or more processors may comprise any one or more of microcontrollers, microprocessors, or embedded processors. The one or more processers may be configured to receive, either directly or indirectly via the control computing device 144, the data from the one or more sensors 129, 134-142, 154, 156 and determine whether to initiate, terminate, adjust, and/or continue providing one or more of electrical stimuli or motorized assistance at the brace 120. For example, the power source may be a battery, such as a rechargeable battery. For example, the communications module may comprise a transmitter, receiver, or transceiver. The communications module may be configured to receive data from one or more of the sensors 129, 134-142, 154, 156. The communications module may be further configured to send instructions to one or more of the motors 128, 152 and/or the electrodes 130A-B, 132, either directly or indirectly via the control computing device 144, to provide motorized assistance and/or electrical stimuli to the paretic leg 104. The control computing device 144 and the user device 148 may communicate via the network device 146.

The system 100 may comprise the network device 146. The network device 146 may comprise a local gateway (e.g., router, modem, switch, hub, access point, combinations thereof, and the like) configured to connect (or facilitate a connection (e.g., a communication session) between) a local area network (e.g., a LAN) to a wide area network (e.g., a WAN). The network device 146 may be configured to receive incoming data (e.g., data packets or other signals) from the brace 120 (e.g., one or more of the sensors 129, 134-142, 154, 156) and route the data to the control computing device 144 and/or the user device 148. The network device 146 may be configured to receive incoming data from the control computing device 144 and/or the user device 148 and route that data to the brace 120 (e.g., one or more of the motors 128, 152 and/or electrodes 130A-B, 132). The network device 146 may be configured to communicate with a network. The network device 146 may be configured for communication with the network via a variety of protocols, such as IP, transmission control protocol, file transfer protocol, session initiation protocol, voice over IP (e.g., VOIP), combinations thereof, and the like. The network device 146 may be configured to facilitate network access via a variety of communication protocols and standards.

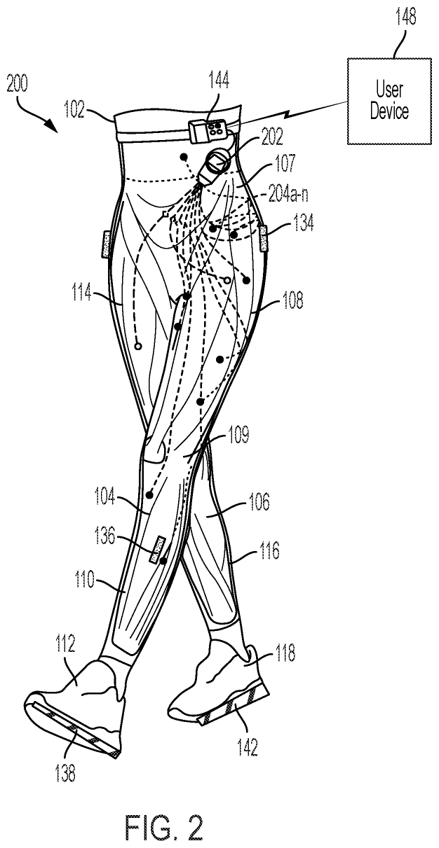

FIG. 2 shows an example system 200 for providing, terminating, or modifying assistance for leg movement. For example, the assistance may be provided to the user 102. While some elements may not be specifically shown, the system 200 of FIG. 2 may comprise the paretic leg 104, non-paretic leg 106, sensors 129, 134-142, 154, 156, user device 148, control computing device 144, and network device 146 as described in FIG. 1. While the example of FIG. 2 shows only one leg of the user 102 is paretic (e.g., the paretic leg 104, and non-paretic leg 106), this is for example purposes only and the user 102 may have both paretic legs (not shown). The system 200 may further comprise a pulse generator 202. The pulse generator 202 may be configured to generate electrical pulses (stimuli) for one or more electrodes 204a-n. All or a portion of the pulse generator 202 may be implanted under the skin of the user 102. For example, the pulse generator 202 may be implanted in the thigh portion 108 of the paretic leg 106. In other examples, the pulse generator 202 may be implanted in another portion of the body of the user 102. For example, the pulse generator 202 may be communicably coupled to the control computing device 144 via wired or wireless communication. For example, the pulse generator 202 may be communicably coupled to the user device 148 via wireless communication. For example, the pulse generator 202 may be electrically coupled to one or more electrodes 204a-n. Each of the one or more electrodes 204a-n may be implanted within a portion of the legs (e.g., the paretic leg 104 and/or the non-paretic leg 106) of the user 102 to provide electrical stimuli to the muscles of the user 102 and/or monitor the activity of the paretic leg 104 and/or non-paretic leg 106 of the user 102. Although it is not shown in FIG. 2, the system 200 applied to the paretic leg 104 may also apply to the other paretic leg (e.g., in case both legs of the user 102 are paretic).

In an example, the actions of neural stimulation by stimulation devices (e.g., electrodes 130A-B, 132) with supplementary motor assistance by motors/actuators (e.g., the motors 128, 152) in a exoskeleton (e.g., the brace 120) may be coordinated/adjusted/controlled for paretic or partially paretic leg movement to maximize contributions of otherwise paralyzed or partially paralyzed or weakened muscles and ensure a biologically inspired ballistic limb trajectory rather than enforcing a predetermined joint angle profile. Thus, the methods, systems, and/or apparatuses described herein may maximize utilization of the user's own muscles and biological energy and gain the benefits of exercising the large lower extremity muscles while allowing the potential for smaller and lighter motorized exoskeleton components. The machine-learning control algorithm may learn to balance muscle and motor contributions during a gait cycle, and may continually adapt to fatigue. The simulation study may instantiate the biologically inspired optimal learning control and prepares for the implementation with backdrivable exoskeletal hardware and implanted neural stimulation systems.

Iterative learning control (ILC) may provide a method for improving the controlled system performance over time. This technique was originally developed for robotics, but has since found diverse application in circuit fabrication, transportation, and even agriculture. ILC may exploit a system that performs repetitive motions and exhibit repetitive measurable errors. ILC may intelligently learn from the errors in the previous iterations to improve performance on the next iteration.

ILC may be used to control hybrid exoskeletons. For example, ILC may treat gait as a repetitive, cyclic system and use ILC methods to achieve coordination between the activated lower limb muscles and the exoskeleton motors. The stimulation on an exoskeleton, neuroprosthesis, hybrid exoskeleton, or brace (e.g., the brace 120) may be modulated by an ILC method on subsequent steps taken by a user based on the control effort of the actuators to track a predefined trajectory. Similarly, ILC may iteratively update the torque produced by the muscles of the user, with the goal of minimizing the interaction torque between the user's limb and the device. The ILC in these applications may not modulate motor torque, which is the responsibility of a higher-level controller. The interaction torque may refer to the torque of force that is exerted between the user (e.g., the wearer) and the exoskeleton, neuroprosthesis, hybrid exoskeleton, or brace (e.g., the brace 120). The interaction torque may arise from the mechanical coupling between the user's body and the exoskeleton, neuroprosthesis, hybrid exoskeleton, or brace (e.g., the brace 120). As the user moves, the exoskeleton's joints and actuators may generate torques to support or augment the movements of the leg(s).

In other examples, the ILC may be used to estimate system dynamics to inform the control computing device 144 (e.g., a sliding-mode controller). The system (e.g., the systems 100-350) may then switch between motor or muscle to control the joint depending on an estimate of fatigue. The system (e.g., the systems 100-350) may also both motor and muscle (e.g., a little motor assistance and more muscle assistance or vice versa) to control the joint depending on an estimate of fatigue. This method may be extended with artificial intelligence or machine-learning techniques such as a neural network-based ILC applied to learn the system dynamics.

As described above, the methods (e.g., methods 900-1100, biologically inspired optimal terminal iterative learning control (BIOTILC) methods) may control and coordinate the muscles and actuators of the exoskeleton, neuroprosthesis, hybrid exoskeleton, or brace (e.g., the brace 120, a motor-assisted hybrid neuroprosthesis (MAHNP)). The exoskeleton, neuroprosthesis, hybrid exoskeleton, or brace (e.g., the brace 120, MAHNP) may combine neuromuscular stimulation with exoskeletal bracing having backdrivable hip and knee joints with a gear mechanism such as harmonic drive transmissions. The methods (e.g., the methods 900-1100, the BIOTILC methods) may be a model-free optimal control method that improves its control performance over time, requires no prior system identification, and is able to dynamically and simultaneously allocate torque across the muscles crossing several degrees-of-freedom and exoskeletal actuators over each step. The methods (e.g., the methods 900-1100, the BIOTILC methods) may maximize muscle recruitment, and therefore the physiological benefits of exercise, with the motors assisting-as-needed to achieve a biologically-inspired ballistic swing limb motion.

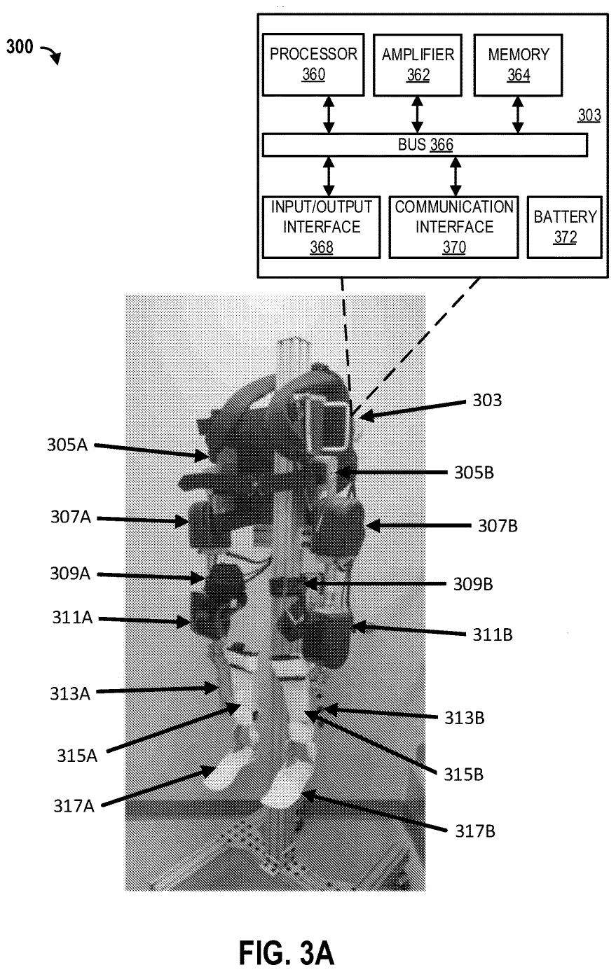

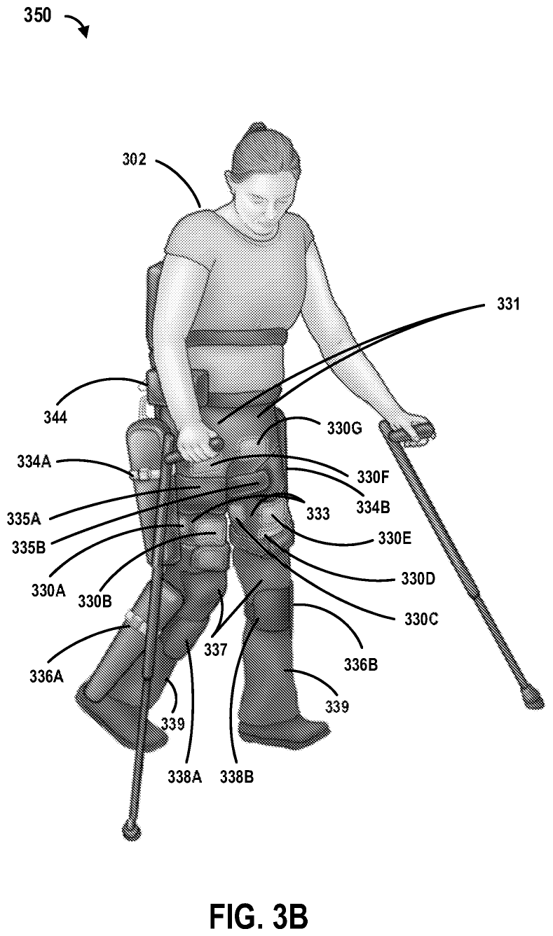

FIG. 3A shows an example system or brace 300 for providing mechanical and/or electrical assistance for paretic or partially paretic leg movement. The brace 300 may be referred to as a neuroprosthesis (e.g., MAHNP), exoskeleton, or hybrid exoskeleton. The brace 300 may comprise multiple components (e.g., of the system 100) described in FIG. 1 for a single or pair of paretic legs. As shown in FIG. 3A, the brace 300 may comprise an electronics housing 303, two trunk orthoses 305A-B, two hip actuators 307A-B, two thigh straps 309-B, two knee actuators 311A-B, two ankle foot orthoses 313-B, two shank portions 315-B, and two foot sections 317-B. The brace 300 may comprise one or more motorized joints. For example, the brace 300 may include two motorized hip joints (e.g., two hip actuators 307A-B) and/or two motorized knee joints (e.g., the two knee actuators 311A-B). For example, each of the actuators 307A-B, 311A-B may be capable of a peak torque of 36 Nm. For example, each of the actuators 307A-B, 311A-B may require less than 6 Nm at all joint speeds (0 to 220°/s) for the corresponding limb to overcome its passive resistance and backdrive the joint. The actuators 307A-B, 311A-B may inject power according to a feedforward model to overcome the internal viscous damping, making the joints of the user or the joints of the brace 300 act as if nearly frictionless, allowing the contracting muscles to drive a leg or one or more portions of the leg with the brace 300 retaining the ability to assist-as-needed. Solenoid mechanisms may lock all joints during quiet standing or solely the knee joint during single stance to allow the muscles to rest. Although it is not shown in FIG. 3A, the brace 300 may further comprise sensors (e.g., the sensors 129, 134-142, 154, 156), additional motors (e.g., motors 128, 152), and/or electrodes (e.g., electrodes 130A-B, 132). The brace 300 may record its internal state, including joint kinematics, and broadcast the data wirelessly to be stored on the electronic housing 303, the control computing device 144 and/or the user device 148 (e.g., a client smartphone, a laptop computer).

The electronics housing 303 may comprise electronic components such as a processor 360, an amplifier 362, memory 364, input/output interface 368, communication interface 370, and a battery 362 as shown in FIG. 3A. The electronic housing 303 may be a backpack or be mounted in a backpack.

The electronics housing 303 may be coupled to the two hip actuators 307A-B and the two knee actuators 311-B. For example, the electronic housing 303 may comprise the control computing device 144 in FIG. 1 communicably coupled to the sensors 129, 134-142, 154, 156, the motors 128, 152, and/or the electrodes 130A-B, 132. The electronic components in the electronics housing 303 may interface with a neuromuscular stimulator controller board (not shown). For example, the neuromuscular stimulator controller board may connect with any combination of two 4-channel surface stimulation boards, 12-channel percutaneous boards, and/or radio frequency boards to control 12- or 16-channel implantable stimulator telemeters. The hip actuators 307A-B may be connected to a fitted, reinforced thoracic-lumbo-sacral orthotic corset, molded to the user's physique. The lower extremities (e.g., legs) of the user may be secured in place with straps along the thigh of the user (e.g., the thigh straps 309A-B) and the system 300 may be connected to the shank of the user (e.g., the shank portions 315A-B) via a metal, straps, a mold, or the like.

As shown in FIG. 3A, The bus 366 may include a circuit for connecting the aforementioned elements 360 to 372 to each other and for delivering communication (e.g., a control message and/or data) between the aforementioned elements 360 to 372. For instance, the bus 366 may be designed to send the signals or sensor data from the processor 360 to the communication interface 370 in order to further transmit the signals or sensor data to an external device such as the user device 148.

The processor 360 may include one or more of a Microcontroller Unit (MCU), a Central Processing Unit (CPU), an Application Processor (AP), or a Communication Processor (CP). The processor 360 may control, for example, at least one of the other constitutional elements of the brace 300 and/or may execute arithmetic operations or data processing for communication. The processing (or controlling) operation of the processor 360, according to various embodiments is described in detail with reference to the following drawings.

For example, the processor 360 may provide a first quantity of electrical stimuli to a paretic leg during the first step of a walking motion. The processor 360 may provide motorized assistance at a first quantity of torque for the paretic leg during the first step of the walking motion. The processor 360 may receive data associated with the paretic leg during the first step of the walking motion. The processor 360 may determine a deviation of the paretic leg from a target configuration. The deviation may be determined during the first step of the walking motion. The processor may modify the first quantity of electrical stimuli during the next step of the walking motion for the paretic leg based on the deviation. Alternatively or additionally, the processor 360 may modify the first quantity of torque provided by the motorized assistance during a next step of the walking motion for the paretic leg based on the deviation.

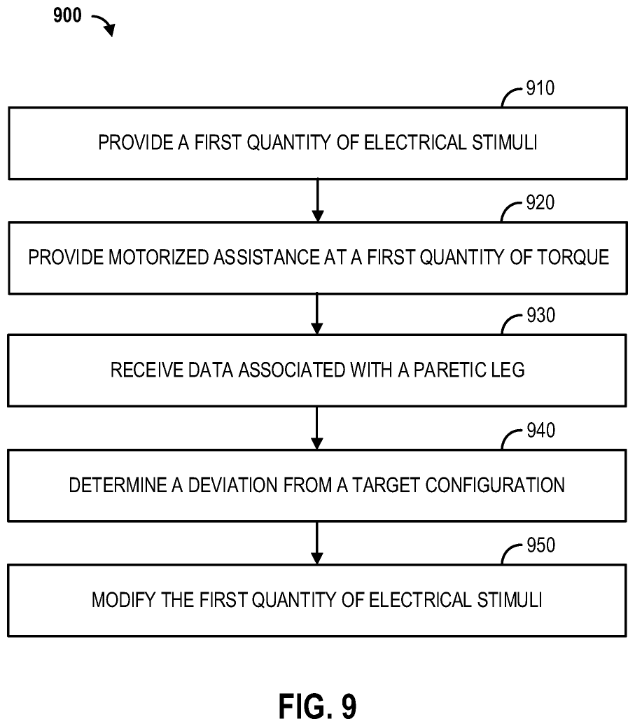

The amplifier 362 may include an instrumentation amplifier such as a MAX4208. An amplifier 362 may be used to amplify the signal received from other devices. The memory 364 may include a volatile and/or non-volatile memory. The memory 364 may store, for example, a command or data related to at least one different constitutional element of the brace 300. According to various example embodiments, the memory 364 may store a software and/or a program. The program may include, for example, a kernel, a middleware, an Application Programming Interface (API), and/or an application program (or an “application”), or the like, configured for controlling one or more functions of the brace 300 and/or an external device. At least one part of the kernel, middleware, or API may be referred to as an Operating System (OS). The memory 364 may include a computer-readable recording medium having a program recorded therein to perform the methods described in FIGS. 9-12 according to various embodiments by the processor 360.

The input/output interface 368 may play a role of an interface for delivering an instruction or data input from a user or a different external device(s) to the different elements of the brace 300. For example, a quantity of electrical stimuli to a paretic leg or a modified quantity of electrical stimuli to a paretic leg may be delivered to one or more electrodes (not shown) (e.g., the electrodes 130A-B, 132) via the input/output interface 368. A quantity of torque or a modified quantity of torque may be delivered to one or more motors (e.g., the motors 128, 152) or actuators 307A-B, 311A-B via the input/output interface 368. Sensor data may be received from one or more sensors (e.g., the sensors 129, 134-142, 154, 156) may be received via the input/output interface 368. Further, the input/output interface 160 may output an instruction or data received from the different element(s) of the brace 300 to the different external device.

The communication interface 370 may establish, for example, communication between the brace 300 and an external device (e.g., the user device 148). For example, the communication interface 370 may communicate with the user device 148 through wireless communication or wired communication.

In another example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication. The near-distance communications may include, for example, at least one of Bluetooth, Wireless Fidelity (WiFi), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication interface may include, for example, at least one of Universal Serial Bus (USB), High Definition Multimedia Interface (HDMI), Recommended Standard-232 (RS-232), power-line communication, Plain Old Telephone Service (POTS), and the like.

FIG. 3B shows an example system 350 for electrical assistance for paralyzed or paretic leg movement. As shown in FIG. 3B, one or both legs of a user 302 may be paralyzed or paretic. The system 350 may be referred to as a neuroprosthesis, exoskeleton, hybrid exoskeleton, or brace (e.g., the brace 120). As shown in FIG. 3B, the system 350 may comprise a control computing device 344, thigh sensors 334A-B, shank sensors 336A-B, electrodes 330A-F, thigh straps 335A-B, and shank straps 338A-B. The control computing device 344 (e.g., the control computing device 144, the electronic housing 303) may be communicably coupled to the sensors 334A-B, 336A-B, the electrodes 330A-F and/or motors (e.g., the motors 128, 152, the actuators 307A-B, 311A-B) (not shown). The control computing device 344 may communicate with the sensors 334A-B, 336A-B, the electrodes 330A-F, and/or the motors via wired or wireless communication. The control computing device 344 may be configured to receive data from the sensors 334A-B, 336A-B and determine whether to initiate, terminate, adjust, and/or continue providing one or more of electrical stimuli at the system 350. For example, the power source of the control computing device 344 may be a battery, such as a rechargeable battery. The control computing device 344 may be configured to send instructions to the electrodes 330A-F to provide electrical stimuli to one or both legs that are paretic or partially paretic.

The electrodes 330A-F may be configured to provide electrical stimuli to the muscles of the pelvic portion, thigh portion and/or any other portion or portions of one or both paretic legs of the user 302 in order to provide assistance with rotation and/or movement of the pelvic portion 331, thigh portion 333, knee portion 337, and/or shank portion 339 of one or both paretic legs during movement. Examples of the muscles for the electrical stimuli may include, but are not limited to, the rectus femoris, vastus lateralis and intermedius, gracilis, sartorius, and tensor fascia latae. The electrodes 330A-F may be operably coupled to the sensors 334A-B, 336A-B. The sensors 334A-B, 336A-B may provide various data indicating measurements of sensing portions of one or both paretic legs. The data may include, but are not limited to, the amount of rotation, acceleration, velocity, angular velocity, orientation, position, and muscle activity of that particular portion of the user's paretic leg. The system 350 may comprise multiple components (of the system 100) described in FIG. 1 for both paretic legs. Although it is not shown, the system 350 may also include multiple components of the system 100 shown in FIG. 1 and/or the system 200 shown in FIG. 2.

A simulation of the biological and mechanical subsystems for mechanical and/or electrical assistance for prosthetic leg movement may be developed, for example, using the OpenSim musculoskeletal modeling software suite. The simulation may comprise a single leg in the swing phase connected to a pelvis fixed in space. The biological aspect of the simulation may include, but are not limited to, an anatomically realistic lower extremity skeleton and all relevant muscle groups that are routinely accessible to percutaneous or surface stimulation in our subjects. The physiological parameters may be determined based on a subject-specific model that reflects the user who has a spinal cord injury (e.g., a T4 motor and sensory complete injury). The muscles typically available for stimulation, and for this user in particular, may be the rectus femoris, vastus lateralis and intermedius, gracilis, sartorius, and tensor fascia latae. The simulation may incorporate the relevant masses, inertia, viscous damping, friction compensation, and torque generation characteristics and limitations of the exoskeletal bracing, details of which are presented in Table 1.

TABLE 1
SIMULATED MAHNP CHARACTERISTICS

| Characteristic | Quantity | Description |
|---|---|---|
| Actuator Masses | 2.2 kg | |
| Actuator Torque Limits | ±36 Nm | Peak torque limit |
| Viscous Damping Model | σ(ω) · \|bω + g\| | Results in < 6 Nm of torque |
| | | required to backdrive actuator |
| | | at joint speeds of ω = 0-220°/s |
| | | [27] |
| Feedforward Friction Compensation | $a \cdot σ ( \frac{ω}{ϕ} ) \cdot \vert b ω + g \vert$ | Compensator derived in [27] |
| Actuator Electrical Current Dynamics | $τ \frac{dc}{dt} + c = s ( t )$ | c is the current, s(t) is the current setpoint, τ = 0.0025 |
| | | resulting in a current rise |
| | | time of 10 ms. |

To control the user's limbs, a neuroprosthesis (e.g., MAHNP) may activate the muscles according to a predefined stimulation pattern, customized to the individual. The muscle activation pattern may depend on the strength and availability of the stimulated muscles. Executing this sequence of varying stimulation pulse widths may recruit the muscles to produce forces on the joints and results in ambulatory motion.

Human gait is often described as “controlled falling”, with the gait patterns naturally taking advantage of passive dynamics to walk in an efficient manner. To begin the swing phase, an impulsive muscle contraction establishes an initial configuration and velocity of the limb. The muscles then relax through the remainder of swing and the leg completes the motion under the influence of momentum and gravity. The relevant muscle groups are then activated in terminal swing to prepare for weight acceptance. However, unlike able-bodied ambulation, walking with stimulation only or with commercially available powered exoskeletons, does not exhibit this ballistic behavior because of limitations in the strength of the atrophied muscle contractions or the enforced trajectory control by the exoskeletal motors. Low passive resistance actuators and powered friction compensation may sufficiently reduce the magnitude of viscous damping to allow a short impulse of torque to produce a passive free-swinging motion in the neuroprosthesis (e.g., MAHNP) under the influence of its own momentum and the force of gravity, similar to a two degree-of-freedom pendulum. This characteristic may make the system amenable to mimicking human gait by programming the motors to produce a burst of flexion torque at the hip and knee for a fixed duration at the beginning of the swing phase to augment the flexor muscles activated via neural stimulation. Once the hip passes a specified flexion threshold, the neuroprosthesis (e.g., MAHNP) may command a knee extension burst to help complete the step and to ensure that stimulation places the limb into the correct position for weight acceptance.

To enhance this biologically inspired burst control with the ability to improve performance over time and iteratively allocate control effort between the muscles and motors, a BIOTILC approach may modulate the torque bursts and the muscle contributions on each step. ILC may be formulated as a repetitive trajectory tracking, with control effort applied throughout the motion providing continuous corrections. The field of Terminal Iterative Learning Control may be a formulation of ILC where the main objective is controlling the endpoint of the iteration and not maximizing the trajectory performance. This formulation may be applied to systems where the start and end points are specified and there is no constraint on how the system traverses between the two points. Alternatively or additionally, it can be applied to systems where it is impossible to record or estimate the states that occur between the start and the end points. BIOTILC may not impose a full trajectory constraint on the neuroprosthesis (e.g., MAHNP) and update the system to enhance the passive portion of swing.

Thus, the neuroprosthesis (e.g., MAHNP) may achieve an appropriate lower extremity configuration to accept weight at the end of each step. Additionally, knee flexion may reach a sufficient level during swing to ensure floor clearance. To achieve this objective, the controller (e.g., the controller computing device 144) may update the stimulation as well as the initial motor bursts. The stimulation patterns may be augmented with scaling factors applied to the baseline pulse widths generated by the neuromuscular stimulators (e.g., the electrodes 130A-B, 132, 204a-n, 330A-F). The muscles in the pattern corresponding to hip flexion, knee flexion, and knee extension may be grouped, and each of these given a corresponding scaling factor. The three scaling factors are α_{hf}, α_{kf}, and αₖₑ, which are the hip and knee flexion and extension scaling factors, respectively.

For example, three torque impulses may be commanded to the actuators (e.g., the actuators 307A-B, 311A-B) for a fixed duration: two flexion torque impulses at the hip and knee at the beginning of swing, τ_{hf} and τ_{kf}, and one knee extension torque, τₖₑ, once the hip has passed a programmed angular threshold. The burst amplitudes of the motors may be modulated and the scaling factors may be applied to the relevant part of the stimulation pattern over each step simultaneously. Each swing phase may be considered an iteration, with the goal of achieving a desired angular configuration y_{d}=[y_{hfd},y_{kfd},y_{ked}]^{T} at a specified time t_{f}. The terminal error may be defined as the deviation of the desired values y_{d} from actual values y at terminal time t_{f}.

The iterative learning control may formulate an optimal control to account for the redundant actuators of the system. Based on Data-Driven Optimal Terminal Iterative Learning Control (DDOTILC), a terminal cost function may be specified that penalizes terminal error at time t_{f} and the rate of change of the input uₖ=[τ_{hf},τ_{kf},τₖₑ,α_{hf},α_{kf},αₖₑ]^{T} over each iteration, with k being the iteration index. Two novel LASSO terms may be added to this cost function to ensure the “muscle first” philosophy—the first of which minimizes the control effort of the motors, the second of which maximizes the recruitment of the muscles.

$$J ( {u}_{k} ) = {‖ {e}_{k} ‖}^{2} + λ {‖ {u}_{k} - {u}_{k - 1} ‖}^{2} + γ \vert {x}_{mot} \cdot {u}_{k} \vert + \frac{β}{\vert {x}_{mus} \cdot {u}_{k} \vert} \qquad Equation ( 1 )$$

In equation (1), eₖ=y_{d}−y may be the terminal error at the end of iteration k. λ may be a weighting factor that limits the magnitude of change of uₖ between iterations. γ may be a weighting factor that governs minimizing the motor contribution, as opposed to the β term which governs maximizing muscle contribution. The scaling factors α may be defined to be greater than zero to prevent the β term from being undefined xₘₒₜ=[1, 1, 1, 0, 0, 0]^{T}, and xₘᵤₛ=[0, 0, 0, 1, 1, 1]^{T} may be vectors which extract the motor and muscle components from uₖ, respectively.

The optimal control law may have an adaptive learning gain that is a function of an estimate of the partial derivatives of the output γ with respect to the inputs uₖ. This gradient estimate may be defined as

$$Δ {\hat{y}}_{k} = {\hat{Ψ}}_{k} Δ {u}_{k} \qquad Equation ( 2 )$$

In equation (2), Δŷ=ŷₖ−yₖ₋₁ may be the estimate of the change in output relative to change in input Δuₖ=uₖ−uₖ₋₁. \hat{Ψ}∈ [symbol] ^{3×6} may be the online estimate of the gradient. The following cost function may be defined to develop an update law to minimize the error between estimate change in outputs Δŷ and the actual change in outputs Δy:

$$J ( {\hat{Ψ}}_{k} ) = {‖ Δ {y}_{k - 1} - {\hat{Ψ}}_{k} Δ {u}_{k - 1} ‖}^{2} + μ {‖ {\hat{Ψ}}_{k} - {\hat{Ψ}}_{k - 1} ‖}^{2} \qquad Equation ( 3 )$$

In equation (3), μ may be a weighting term that minimizes the rate of change in the iterative estimate. The iterative gradient update law may be computed by taking the partial derivative of the cost function with respect to \hat{ψ}ₖ and setting it to zero. This system \hat{ψ}ₖ may result in equation (4):

$${\hat{Ψ}}_{k} = {\hat{Ψ}}_{k - 1} + \frac{η ( Δ {y}_{k - 1} - {\hat{Ψ}}_{k - 1} Δ {u}_{k - 1} ) Δ {u}_{k - 1}^{T}}{μ + {‖ Δ {u}_{k - 1} ‖}^{2}} \qquad Equation ( 4 )$$

In equation (4), η may be a learning gain that dictates how much the estimate changes due to estimation error over each iteration. The terminal cost function may be then rewritten to incorporate the estimate \hat{ψ}ₖ:

$$J ( {u}_{k} ) = {‖ {e}_{k - 1} - {\hat{Ψ}}_{k} ( {u}_{k} - {u}_{k - 1} ) ‖}^{2} + λ {‖ {u}_{k} - {u}_{k - 1} ‖}^{2} + γ \vert {x}_{mot} \cdot {u}_{k} \vert + \frac{β}{\vert {x}_{mus} \cdot {u}_{k} \vert} \qquad Equation ( 5 )$$

The partial derivative of the cost function may be taken with respect to uₖ and set to zero. Solving for uₖ may result in the optimal terminal iterative learning control equation:

$${u}_{k} = {u}_{k - 1} + \frac{ρ ( {Ψ}_{k}^{T} {e}_{k - 1} )}{{‖ {Ψ}_{k} ‖}^{2} + λ} - \frac{γ {x}_{mot} sgn ( {x}_{mot} \cdot {u}_{k} )}{2 ( {‖ {\hat{Ψ}}_{k} ‖}^{2} + λ )} + \frac{β {x}_{mus}}{{x}_{mus} \cdot {u}_{k} \vert {x}_{mus} \cdot {u}_{k} \vert} \cdot \frac{1}{2 ( {‖ {\hat{Ψ}}_{k} ‖}^{2} + λ )} \qquad Equation ( 6 )$$

For the first iteration, an estimate of the gradient \hat{Ψ}₀ may be needed. In practice, only knowledge of the signs of the elements of \hat{Ψ}₀ may be needed. To ensure that actual hardware realized this in real-time, the noncausal uₖ terms in the dot products on the right-hand side of the update law may be replaced with uₖ₋₁ when implemented. Finally, \hat{ψ}ₖ may be reset to \hat{Ψ}₀ if the signs of the elements of \hat{ψ}ₖ no longer match those of the initial estimate.

In an example, a desired hip flexion of y_{hfd}=30° and a desired knee extension of y_{ked}=0° at time t_{f} may be specified as a terminal configuration to ensure an appropriate limb orientation to accept weight. To guarantee floor clearance, the desired maximum knee flexion may be y_{kfd}=50° during swing. The duration of the step was t_{f}=0.5 s, and the stimulation patterns may be compressed accordingly. The flexion torque bursts commanded to the motors may last for 0.2 s from the onset of swing, and the knee extension burst, or late swing burst, may last for 0.2 seconds. This latter burst may be executed once the hip exceeded a specified threshold of 12° of flexion.

The following constants: ρ−0.6, β=0.8, γ=0.5, λ=0.1, μ=1, η=0.2, may be applied to BIOTILC. The value of ρ may be derived heuristically by first isolating its effect on the terminal error across iterations by setting the β and γ terms to zero. ρ may then be set to zero and slowly increased, which may result in increasing terminal error convergence rate. The system may exhibit oscillations in the terminal error once ρ was too large, never reaching a stable, minimal error. At that point, ρ may be reduced to the last value that had the highest convergence rate while producing a stable, minimal terminal error. β and γ may then be tuned to have tangible effects on maximizing muscle recruitment and minimizing motor control effort, while following a similar tuning routine as described above. An additional stop criterion was when these terms caused a constant offset in error due to the system producing less torque than possible. μ η, and λ may be rate-limiting terms to ensure stable estimate of \hat{Ψ} and uₖ, and thus may be set to default values. Further tuning of these latter constants may provide a higher convergence rate, however there is an increased likelihood of poor performance due to incorrect gradient estimates. BIOTILC may be initialized with the following gradient estimate:

$${\hat{Ψ}}_{0} = [ \begin{matrix}1 & 0 & 0 & 1 & 0 & 0 \\ 0 & 1 & 0 & 0 & - 1 & 0 \\ 0 & 0 & 1 & 0 & 0 & 1\end{matrix} ]$$

This estimate assumes only redundant actuation and no coupling across joints. For example, it assumes that both the hip motor and the hip flexion muscle stimulation scaling factor influence the amount of terminal hip flexion, whereas the hip flexion torque does not affect the amount of knee flexion. However, if there is coupling between the two terms, BIOTILC may determine the existence and magnitude of the coupling term and account for it automatically.

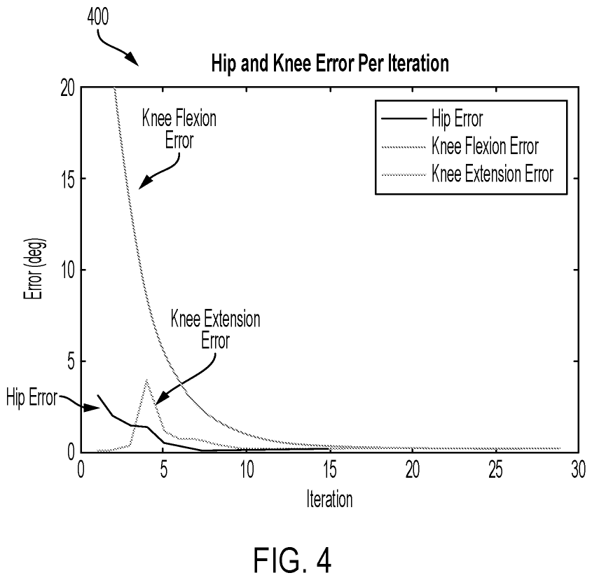

FIG. 4 shows an example graph 400 for hip and knee errors per iteration. The performance of BIOTILC at achieving the learning objective over thirty iterations or steps is shown in FIG. 4. For the first iteration, the motors were commanded zero torque, and all stimulation scaling factors were set to one, meaning that the simulated neuroprosthesis (e.g., MAHNP) may be driven purely by recruited muscle movement via the original stimulation pattern. By the 15^{th} iteration, the system had adapted by maximizing muscular recruitment and assisting as needed with motor bursts to minimize the error. BIOTILC is capable of both ensuring foot clearance as well as attaining the terminal stance configuration required to accept weight.

After 30 iterations, the estimate of BIOTILC may be:

$${\hat{Ψ}}_{30} = [ \begin{matrix}1.09 & - 0.02 & - 0.07 & 1.67 & 0.6 & 0.52 \\ - 0.07 & 1.69 & - 0.02 & 0 & - 1.03 & 0.04 \\ - 0.01 & 0.07 & 1.05 & - 0.05 & - 0.05 & 1.01\end{matrix} ]$$

The most significant joint coupling terms may be in row 1, columns 5 and 6 of 0.6 and 0.52, respectively. These terms may indicate that the change in terminal hip flexion is affected not just by the hip motor (1.09) and hip flexion stimulation scaling factor (1.67), but by an increase in the stimulation scaling factors governing knee flexion (0.60) and knee extension (0.52) as well. One of the muscles recruited for knee flexion may be the gracilis, and for knee extension the rectus femoris may be activated. Both muscles may be biarticular: They affect movement simultaneously across the hip and knee joints. The gracilis may contribute to both knee flexion and hip flexion, while the rectus femoris affects both hip flexion and knee extension. It is clear that BIOTILC estimated the coupling terms that indicate the influence of both of these muscles on hip flexion. The entry in row 2, column 2 may indicate that the knee flexion motor burst had a large influence on achieving the desired angle for floor clearance. This may correlate with the knee flexion burst being the highest commanded torque of all motorized bursts. Finally, there may be small, almost negligible estimated coupling influences for the rest of the terms.

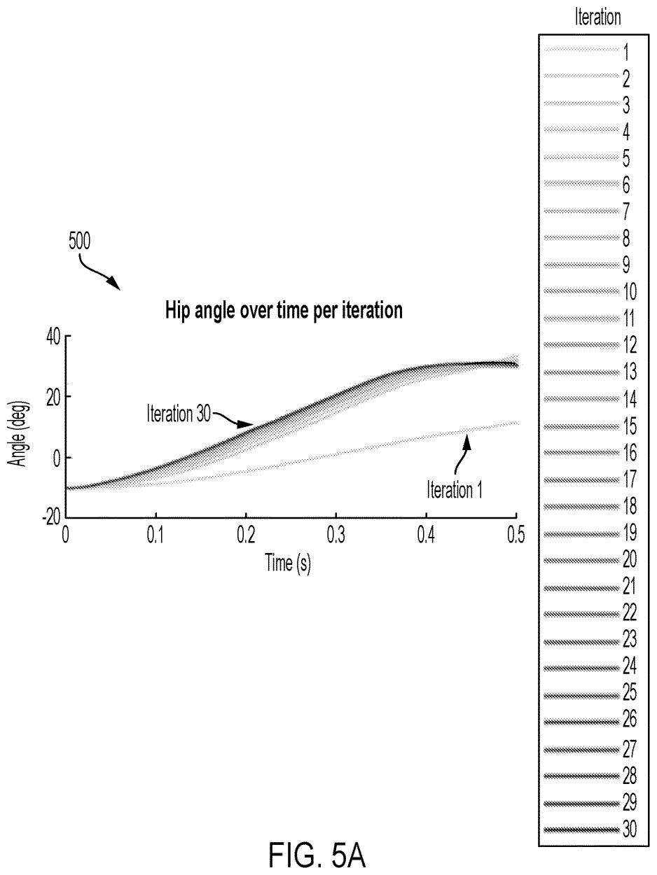

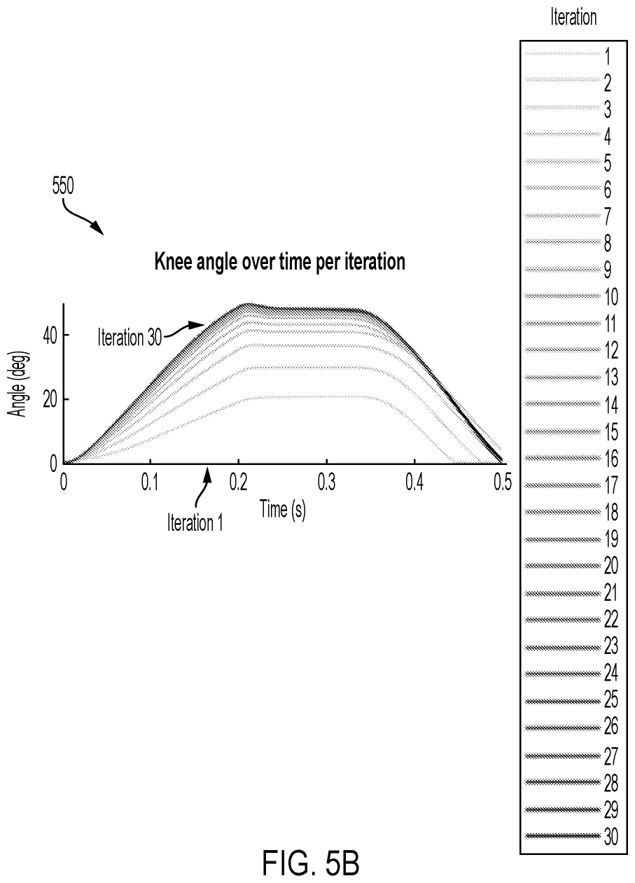

FIG. 5A shows an example graph 500 for hip angle over time per iteration. FIG. 5B shows an example graph 550 for knee angle over time per iteration. As shown in FIG. 5A, joint angle (e.g., hip angle) may progress as the controller (e.g., the control computing device 144) updates input with each successive swing phase. Lines in FIG. 5A from iteration 1 to iteration 30 (e.g., becomes darker) may indicate progressive iterations. As shown in FIG. 5B, joint angle (e.g., knee angle) may progress as the controller (e.g., the control computing device 144) updates input with each successive swing phase. Lines in FIG. 5B from iteration 1 to iteration 30 (e.g., becomes darker) may indicate progressive iterations.

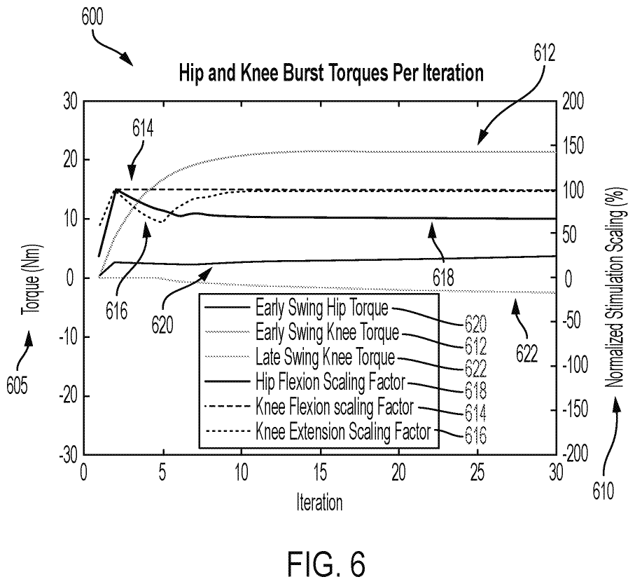

FIG. 6 shows an example graph 600 for hip and knee burst torques per iteration. FIG. 6 shows that the “muscle-first” objective of maximizing muscle recruitment and minimizing motor control effort is achieved. The FIG. 6 shows the torque commanded to the motors, as indicated by lines 612, 620, 622 paired with the left axis 605. The right axis 610 may be paired with lines 614, 616, 618, which represent normalized stimulation scaling factors. A scaling of 100% may indicate that the multiplicative scaling factor applied to the pattern resulted in the pattern having a peak pulse-width of 255 microseconds, the maximum the stimulator control board can output. The stimulation scaling factor for knee flexion maximized within the first 5 iterations, and the knee extension scaling factor saturated within 15 iterations. The hip flexion scaling factor maximized within 5 iterations, but on further steps BIOTILC determined that with the hip motor, hip flexors such as the sartorius and tensor fascia latae, gracilis, and rectus femoris acting on the joint, it was unnecessary to maximize hip flexor muscle recruitment to achieve the learning objective. The motors made up for any deficits, with 21.1 Nm of flexion torque required from the knee motor to achieve foot clearance. To achieve the terminal configuration only 3.6 Nm hip flexion torque and 2.5 Nm knee extension torque was required of the motors. None of the motors reached maximal peak torque of 36 Nm, while the stimulation scaling factors aside from the hip scaling factor were near maximal.

To test BIOTILC's ability to adapt to muscular fatigue, a simple worst-case fatigue model may be applied to the system. For this simulation, the BIOTILC weighting terms may be identical to those described above. The initial inputs u₀ and gradient estimation \hat{Ψ}₀ instantiated with the learned values from the 30^{th} iteration of the previous simulation may be u₃₀ and \hat{Ψ}₃₀, respectively. For the first five of this simulation, muscle torque output is decreased by 10%. The system reached 50% strength on the 5^{th} step and remained at that level for all subsequent steps.

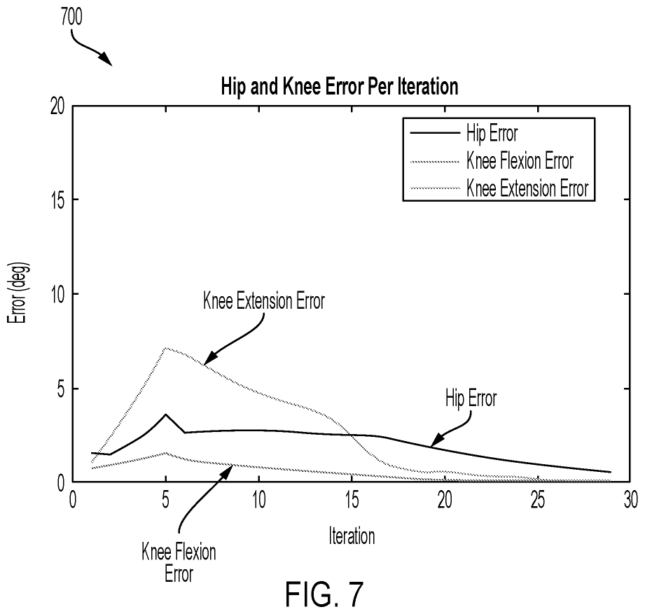

FIG. 7 shows an example graph 700 for hip and knee errors per iteration. The hip and knee errors in FIG. 7 may be the absolute terminal error over each iteration in the presence of simulated fatigue. In the first five steps, the terminal error increased as the muscles weakened. The terminal error peaked at 3.6°, 1.6°, and 7° for the hip flexion, knee flexion, and knee extension errors, respectively. Once the system experienced constant fatigue, BIOTILC adapted, and by the 30^{th} iteration there was less than 0.6° of error for each desired terminal configuration.

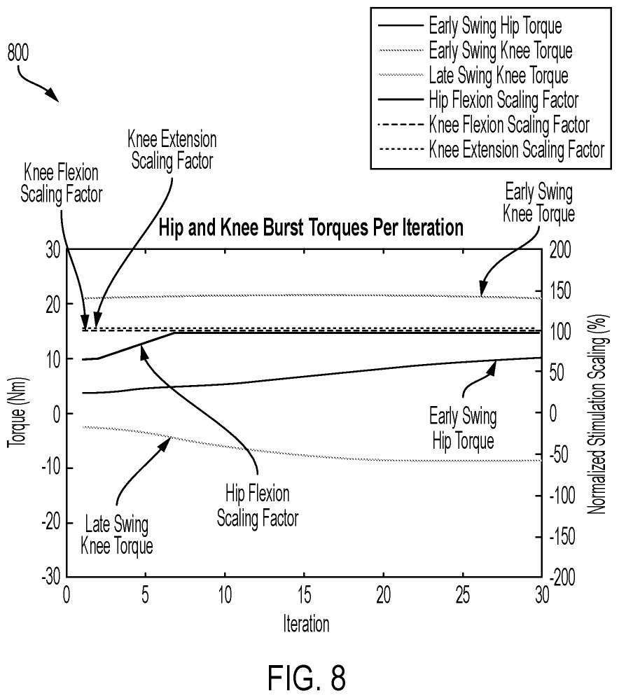

FIG. 8 shows an example graph 800 for hip and knee burst torques per iteration. As shown in FIG. 8, control effort may be distributed across muscular recruitment and motor burst torques over each iteration. Exhibiting the “muscle-first philosophy”, the rate of increase in hip flexion muscular recruitment is faster than the increase in motorized burst torque, becoming maximized at the 7^{th} iteration. Motorized knee flexion torque stayed largely the same, with the motorized hip flexion and knee extension torques reaching 10.12 Nm and 8.8 Nm, respectively, on the 30^{th} iteration. FIG. 8 shows BIOTILC's ability to adapt to the presence of fatigue in the system.

In an example, the constants derived for the simulation may be transferred to the physical system with little to no changes or further tuning. However, some tuning may be needed since the simulation cannot capture all the subtle dynamics of the physical system. In other examples, the signs of the initial gradient estimate may be needed, and some of the weighting factors may be re-tuned to ensure convergence and prevent oscillation about the terminal configuration over each iteration.

BIOTILC is a cooperative iterative learning controller designed to control a “muscle-first” motor-assisted hybrid neuroprosthesis that combines neuromuscular stimulation and motorized actuation. It is a model-free method capable of iteratively improving performance by maximizing the recruitment of the muscles and minimizing the motors simultaneously over each step. Simulations of the neuroprosthesis (e.g., MAHNP) in swing phase show the efficacy of this method in achieving the correct terminal swing configuration, ensuring foot clearance, and exemplifying the “muscle-first” paradigm, as well as adapting to muscular fatigue.

In an example, the stimulation patterns may be iteratively learned for each muscle over time, as opposed to scaling a pre-existing pattern. Additionally, the single stance limb as well as swing limb may be controlled to drive the system forward. The simulation itself can be enhanced by incorporating the effect of heel strike, as well as removing the fixed constraint on the pelvis and allowing it to move through space to represent natural forward progression of the body.

It is noted that an exoskeletal assist-as-needed paradigm may need a forgetting factor applied to the motorized portion of the system to keep the pilot challenged. Thus, BIOTILC can be reformulated with a forgetting factor based on an extension of Data-Driven Optimal Terminal Iterative Learning Control (DDOTILC). Additionally, it is possible to extend the optimal control method with higher order learning terms, control of multiple intermediate pass points, and initial value dynamic compensation.

FIG. 9 shows an example method 900 for providing electrical and/or mechanical assistance for leg movement. The method 900 may be performed by any device such as the control computing device 144, 344, the user device 148, and a computing device in communication with the one or more sensors 129, 134-142, 154, 156, the one or more motors 128, 152, and/or the one or more electrodes 130A-B, 132. Although the method 900 is described for a paretic leg (e.g., the paretic leg 104), the method 900 may also apply to the other paretic leg when both legs of a user (e.g., the user 102) are paretic.

At step 910, a first quantity of electrical stimuli may be provided to a paretic leg during the first step of a walking motion. For example, a computing device (e.g., the control computing device 144, 344) may provide the first quantity of electrical stimuli to one or more portions of a paretic leg during the first step of the walking motion. The computing device may provide the first quantity of electrical stimuli based on one or more electrodes (e.g., the one or more electrodes 130A-B, 132). The one or more electrodes may be positioned at one or more portions along the outer surface of the paretic leg (e.g., the pelvic portion 107, the thigh portion 108, the knee portion 109, the shank portion 110). The one or more electrodes may be configured to provide electrical stimuli to the muscles of the paretic leg in order to provide assistance with rotation and/or movement of the paretic leg during movement. For example, the electrical stimuli may be provided to the rectus femoris, vastus lateralis and intermedius, gracilis, sartorius, and tensor fascia latae of the paretic leg.

The first quantity of electrical stimuli may indicate the baseline amount of electrical stimulation (e.g., predefined electrical stimulation pattern). The first quantity of electrical stimuli may indicate the length, amplitude, and/or frequency of the electrical stimulation. The first quantity of electrical stimuli may be determined based on the strength and/or availability of the muscles of the paretic leg. The first quantity of electrical stimuli may cause a contraction of a muscle in the one or more portions of the paretic leg. For example, the first quantity of electrical stimuli may recruit the muscles (e.g., inactive muscles) to force the first step of the walking motion. To provide the first quantity of electrical stimuli, the computing device (e.g., the control computing device 144, 344) may communicate with the one or more electrodes via wired or wireless communication. For example, the computing device may communicate wirelessly via one or more of Wi-Fi, Bluetooth, Bluetooth Low Energy (BLE), Zigbee, or any other known wireless protocol with the one or more electrodes. The term quantity of electrical stimuli may be interchangeably used with stimulus parameters throughout this disclosure. For example, the first quantity of electrical stimuli may be interchangeably used with the first stimulus parameters.

At step 920, motorized assistance may be provided for the paretic leg at a first quantity of torque during the first step of the walking motion. For example, the computing device (e.g., the control computing device 144, 344) may provide the motorized assistance for the paretic leg at the first quantity of torque during the first step of the walking motion. The computing device may provide the motorized assistance based on one or more motors (e.g., the one or more motors 128, 152, 307A-B, 311A-B). The one or more motors may be positioned at or near one or more joints of the paretic leg (e.g., between the thigh section 122 and the shank section 124 and/or between the thigh section 122 and the hip section 150). The motorized assistance may be provided by activating the one or more motors coupled to the paretic leg. For example, the one or more motors 128, 152 of the brace 120 or the one or more actuators 307A-B, 311A-B of the exoskeleton 300 may be activated at the first quantity of torque to provide motorized assistance for the paretic leg. To provide the motorized assistance for the paretic leg, the computing device (e.g., the control computing device 144, 344) may communicate with the one or more motors via wired or wireless communication. For example, the computing device may communicate wirelessly via one or more of Wi-Fi, Bluetooth, Bluetooth Low Energy (BLE), Zigbee, or any other known wireless protocol with the one or more motors. It is noted that the first quantity of electrical stimuli and at least a portion of the motorized assistance at the first quantity of torque may be provided/performed simultaneously.

At step 930, data associated with the paretic leg during the first step of the walking motion may be received. For example, the computing device (e.g., the control computing device 144, 344) may receive data associated with the paretic leg during the first step of the walking motion. The data associated with the paretic leg during the first step of the walking motion may be received from one or more sensors (e.g., the one or more sensors 129, 134-142, 154, 156). The one or more sensors may comprise a thigh sensor (e.g., the thigh sensor 134), a shank sensor (e.g., the shank sensor 136), a heel-strike sensor (e.g., the heel-strike sensor 138), a foot sensor (e.g., the foot sensor 140), and a hip sensor. The data associated with the paretic leg during the first step of the walking motion may include, but are not limited to, acceleration data, velocity data, angular velocity data, orientation data, muscle activity data, and position data.

The data associated with the paretic leg during the first step of the walking motion may be received via wired or wireless communication. For example, the computing device (e.g., the control computing device 144, 344) may communicate with the one or more sensors via wired or wireless communication such as LAN, Wi-Fi, Bluetooth, Bluetooth Low Energy (BLE), Zigbee, or any other known wired/wireless protocol with the one or sensors.

At step 940, a deviation from a target configuration for the paretic leg during the first step of the walking motion may be determined. For example, the computing device (e.g., the control computing device 144, 344) may determine, based on the data received from the one or more sensors, the deviation from the target configuration for the paretic leg during the first step of the walking motion. The deviation of the paretic leg may comprise a difference between a target angular configuration for the paretic leg and an actual angular configuration for the paretic leg during the first step. For example, the computing device may determine a first position of the portion of the paretic leg during the first step at a first time. The computing device may determine a target position for the portion of the paretic leg during the first step at the first time. The computing device may determine that the first position of the portion of the paretic leg varies from the target position for the portion of the paretic leg at the first time.

In other examples, the computing device may determine a first angular velocity of the portion of the paretic leg during the first step at a first time. The computing device may determine a target angular velocity for the portion of the paretic leg during the first step at the first time. The computing device may determine the difference between the first angular velocity of the portion of the paretic leg and the target angular velocity for the portion of the paretic leg at the first time satisfies a threshold (e.g., angular threshold). Alternatively, or additionally, the deviation of the paretic leg may be determined based on a plurality of weighting factors. The plurality of weighting factors may comprise a first weighting factor minimizing the provision of motorized assistance and a second weighting factor maximizing muscle contribution of the paretic leg by electrical stimuli.

At step 950, the first quantity of electrical stimuli may be modified for the next step of the walking motion of the paretic leg. For example, the computing device (e.g., the control computing device 144, 344) may modify, based on the deviation, the first quantity of electrical stimuli during the next step of the walking motion for the paretic leg. For example, the computing device may increase an amount of the electrical stimuli to a level above the first quantity of electrical stimuli or decrease the amount of the electrical stimuli to a second level below the first quantity of electrical stimuli. Alternatively or additionally, the computing device may increase an amount of time the electrical stimuli are provided to the portion of the paretic leg or decrease the amount of time the electrical stimuli are provided to the portion of the paretic leg. Alternatively or additionally, the computing device may increase a frequency the electrical stimuli are provided to the portion of the paretic leg, or decrease the frequency the electrical stimuli are provided to the paretic leg.

The computing device (e.g., the control computing device 144, 344) may also modify, based on the deviation, the first quantity of torque provided by the motorized assistance during the next step of the walking motion for the paretic leg. During one or more phases of the next step of the walking motion, the computing device may provide the modified quantity of electrical stimuli to the portion of the paretic leg. It is noted that the modified quantity of electrical stimuli and at least a portion of the motorized assistance at the modified quantity of torque may be provided/performed simultaneously. It is also noted that the steps 910-950 described in FIG. 9 may be repeated for the paretic leg during one or more phases of the next step of the walking motion and/or for the other paretic leg during one or more phases of the next step of the walking motion.

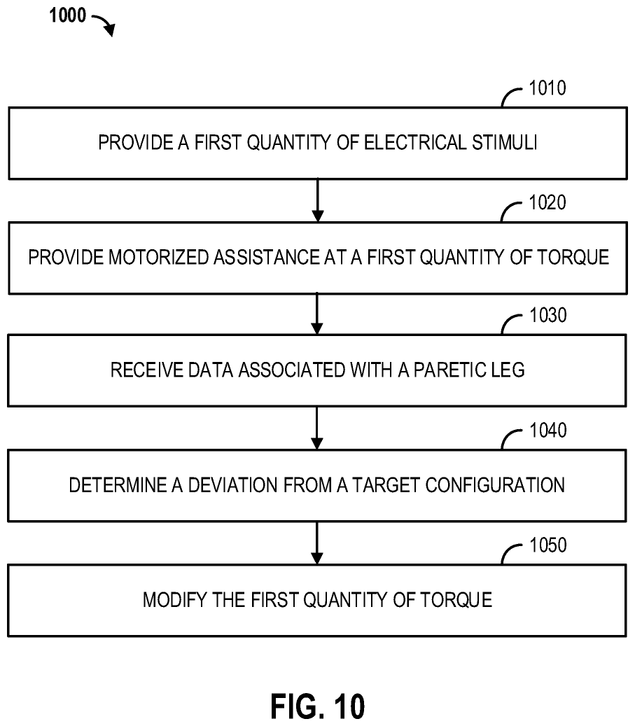

FIG. 10 shows an example method 1000 for providing electrical and/or mechanical assistance for leg movement. The method 1000 may be performed by any device such as the control computing device 144, 344, the user device 148, and a computing device in communication for the one or more sensors 129, 134-142, 154, 156, the one or more motors 128, 152, and/or the one or more electrodes 130A-B, 132. Although the method 1000 is described with a paretic leg (e.g., the paretic leg 104), the method 1000 may also apply to the other paretic leg when both legs of a user (e.g., the user 102) are paretic.

At step 1010, a first quantity of electrical stimuli may be provided to a paretic leg during the first step of a walking motion. For example, a computing device (e.g., the control computing device 144, 344) may provide the first quantity of electrical stimuli to one or more portions of a paretic leg during the first step of the walking motion. The computing device may provide the first quantity of electrical stimuli using one or more electrodes (e.g., the one or more electrodes 130A-B, 132) positioned at one or more portions of the paretic leg (e.g., the pelvic portion 107, the thigh portion 108, the knee portion 109, the shank portion 110). The one or more electrodes may be configured to provide electrical stimuli to the muscles of the paretic leg to provide movement assistance of the paretic leg. The muscles of the paretic leg may be inactive or weak.

The first quantity of electrical stimuli may indicate the length, amplitude, and/or frequency of the electrical stimulation. The first quantity of electrical stimuli may be determined based on the strength and/or availability of the muscles of the paretic leg. The first quantity of electrical stimuli may cause a contraction of a muscle in the one or more portions of the paretic leg. For example, the first quantity of electrical stimuli may recruit the muscles (e.g., inactive muscles) to force the first step of the walking motion. To provide the first quantity of electrical stimuli, the computing device (e.g., the control computing device 144, 344) may communicate with the one or more electrodes via wired or wireless communication. The term quantity of electrical stimuli may be interchangeably used with stimulus parameters throughout this disclosure. For example, the first quantity of electrical stimuli may be interchangeably used with the first stimulus parameters.

At step 1020, motorized assistance may be provided for the paretic leg at a first quantity of torque during the first step of the walking motion. For example, the computing device (e.g., the control computing device 144, 344) may provide the motorized assistance for the paretic leg at the first quantity of torque during the first step of the walking motion. The computing device may provide the motorized assistance using one or more motors (e.g., the one or more motors 128, 152, 307A-B, 311A-B). The computing device may provide the motorized assistance by activating the one or more motors coupled to the paretic leg. To provide the motorized assistance for the paretic leg, the computing device (e.g., the control computing device 144, 344) may communicate with the one or more motors via wired or wireless communication. It is noted that the first quantity of electrical stimuli and at least a portion of providing the motorized assistance at the first quantity of torque may be performed simultaneously.

At step 1030, data associated with the paretic leg during the first step of the walking motion may be received. For example, the computing device (e.g., the control computing device 144, 344) may receive data associated with the paretic leg during the first step of the walking motion. The data associated with the paretic leg during the first step of the walking motion may be received from one or more sensors (e.g., the one or more sensors 129, 134-142, 154, 156). The one or more sensors may comprise a thigh sensor (e.g., the thigh sensor 134), a shank sensor (e.g., the shank sensor 136), a heel-strike sensor (e.g., the heel-strike sensor 138), a foot sensor (e.g., the foot sensor 140), and a hip sensor. The data associated with the paretic leg during the first step of the walking motion may include, but are not limited to, acceleration data, velocity data, angular velocity data, orientation data, muscle activity data, and position data. The data associated with the paretic leg during the first step of the walking motion may be received via wired or wireless communication.

At step 1040, a deviation from a target configuration for the paretic leg during the first step of the walking motion may be determined. For example, the computing device (e.g., the control computing device 144, 344) may determine, based on the data received from the one or more sensors, the deviation from the target configuration for the paretic leg during the first step of the walking motion. The deviation of the paretic leg may comprise a difference between a target angular configuration for the paretic leg and an actual angular configuration for the paretic leg during the first step. For example, the computing device may determine a first position of the portion of the paretic leg during the first step at a first time. The computing device may determine a target position for the portion of the paretic leg during the first step at the first time. The computing device may determine that the first position of the portion of the paretic leg varies from the target position for the portion of the paretic leg at the first time.

In other examples, the computing device may determine a first angular velocity of the portion of the paretic leg during the first step at a first time. The computing device may determine a target angular velocity for the portion of the paretic leg during the first step at the first time. The computing device may determine the difference between the first angular velocity of the portion of the paretic leg and the target angular velocity for the portion of the paretic leg at the first time satisfies a threshold (e.g., angular threshold). Alternatively, or additionally, the deviation of the paretic leg may be determined based on a plurality of weighting factors. The plurality of weighting factors may comprise a first weighting factor minimizing the provision of motorized assistance and a second weighting factor maximizing muscle contribution of the paretic leg by electrical stimuli.

At step 1050, the first quantity of torque may be modified for the next step of the walking motion of the paretic leg. For example, the computing device (e.g., the control computing device 144, 344) may modify, based on the deviation, the first quantity of torque during the next step of the walking motion for the paretic leg. For example, the computing device may increase an amount of torque to a level above the first quantity of torque or decrease the amount of the torque to a second level below the first quantity of torque. Alternatively or additionally, the computing device may increase an amount of time motorized assistance is provided at the first quantity of torque, or decrease the amount of time motorized assistance is provided at the first quantity of torque.

The computing device (e.g., the control computing device 144, 344) may also modify, based on the deviation, the first quantity of electrical stimuli during the next step of the walking motion for the paretic leg. During one or more phases of the next step of the walking motion, the computing device may provide motorized assistance for the paretic leg at the modified quantity of torque. It is noted that at least a portion of the motorized assistance at the modified quantity of torque and the modified quantity of electrical stimuli may be provided/performed simultaneously. It is also noted that the steps 1010-1050 described in FIG. 10 may be repeated for the paretic leg during one or more phases of the next step of the walking motion and/or for the other paretic leg during one or more phases of the next step of the walking motion.

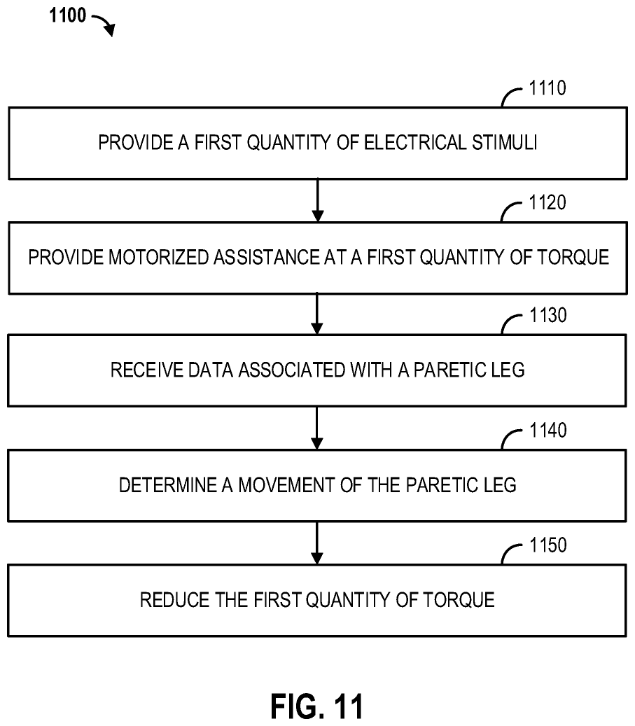

FIG. 11 shows an example method 1100 for providing electrical and/or mechanical assistance for leg movement. The method 1100 may be performed by any device such as the control computing device 144, 344, the user device 148, and a computing device in communication with the one or more sensors 129, 134-142, 154, 156, the one or more motors 128, 152, and/or the one or more electrodes 130A-B, 132. Although the method 1100 is described for a paretic leg (e.g., the paretic leg 104), the method 1100 may also apply to the other paretic leg when both legs of a user (e.g., the user 102) are paretic.

At step 1110, a first quantity of electrical stimuli may be provided to a paretic leg during the first step of a walking motion. For example, a computing device (e.g., the control computing device 144, 344) may provide the first quantity of electrical stimuli to one or more portions of a paretic leg during the first step of the walking motion. The first quantity of electrical stimuli may be provided based on one or more electrodes (e.g., the one or more electrodes 130A-B, 132) positioned at one or more portions of the paretic leg (e.g., the pelvic portion 107, the thigh portion 108, the knee portion 109, the shank portion 110). The one or more electrodes may be configured to provide electrical stimuli to the muscles, such as the rectus femoris, vastus lateralis and intermedius, gracilis, sartorius, and tensor fascia latae, of the paretic leg.

The first quantity of electrical stimuli may indicate the length, amplitude, and/or frequency of the electrical stimulation. The first quantity of electrical stimuli may be determined based on the strength and/or availability of the muscles of the paretic leg. The first quantity of electrical stimuli may cause a contraction of a muscle in the one or more portions of the paretic leg. The computing device (e.g., the control computing device 144, 344) may communicate with the one or more electrodes via wired or wireless communication to provide the electrical stimuli. For example, the computing device may communicate wirelessly via one or more of Wi-Fi, Bluetooth, Bluetooth Low Energy (BLE), Zigbee, or any other known wireless protocol with the one or more electrodes. The term quantity of electrical stimuli may be interchangeably used with stimulus parameters throughout this disclosure. For example, the first quantity of electrical stimuli may be interchangeably used with the first stimulus parameters.

At step 1120, motorized assistance may be provided for the paretic leg at a first quantity of torque during the first step of the walking motion. For example, the computing device (e.g., the control computing device 144, 344) may provide the motorized assistance for the paretic leg at the first quantity of torque during the first step of the walking motion. The motorized assistance may be provided based on one or more motors (e.g., the one or more motors 128, 152, 307A-B, 311A-B) positioned at or near one or more joints of the paretic leg (e.g., between the thigh section 122 and the shank section 124 and/or between the thigh section 122 and the hip section 150). The motorized assistance may be provided by activating the one or more motors coupled to the paretic leg. To provide the motorized assistance for the paretic leg, the computing device (e.g., the control computing device 144, 344) may communicate with the one or more motors via wired or wireless communication. For example, the computing device may communicate wirelessly via one or more of Wi-Fi, Bluetooth, Bluetooth Low Energy (BLE), Zigbee, or any other known wireless protocol with the one or more motors. It is noted that the first quantity of electrical stimuli and at least a portion of the motorized assistance at the first quantity of torque may be provided/performed simultaneously.

At step 1130, data associated with the paretic leg during the first step of the walking motion may be received. For example, the computing device (e.g., the control computing device 144, 344) may receive data associated with the paretic leg during the first step of the walking motion. The data associated with the paretic leg during the first step of the walking motion may be received from one or more sensors (e.g., the one or more sensors 129, 134-142, 154, 156). The data associated with the paretic leg during the first step of the walking motion may include, but are not limited to, acceleration data, velocity data, angular velocity data, orientation data, muscle activity data, and position data. The data associated with the paretic leg during the first step of the walking motion may be received via wired or wireless communication such as Wi-Fi, Bluetooth, Bluetooth Low Energy (BLE), Zigbee, or any other known wired/wireless protocol with the one or sensors.

At step 1140, a movement of the paretic leg that satisfies a target configuration for the paretic leg during the first step of the walking motion may be determined. For example, based on the received data, the computing device (e.g., the control computing device 144, 344) may determine that the movement of the paretic leg during the first step of the walking motion satisfies the target configuration for the paretic leg during the first step of the walking motion. For example, the movement of the paretic leg during the first step of the walking motion may exceed the target configuration for the paretic leg during the first step of the walking motion. The movement of the paretic leg during the first step of the walking motion may be an actual angular configuration for the paretic leg during the first step. The actual angular configuration for the paretic leg during the first step may exceed the target configuration (e.g., a target angular configuration) for the paretic leg. The movement of the paretic leg satisfying the target configuration may be determined based on one or more positions of the paretic leg during the first step of the walking motion. For example, the computing device may determine a first position of the portion of the paretic leg during the first step at a first time and a target position for the portion of the paretic leg during the first step at the first time. The computing device may determine that the first position of the portion of the paretic leg exceeds the target position for the portion of the paretic leg at the first time.

At step 1150, the first quantity of torque provided by the motorized assistance during a second step of the walking motion for the paretic leg may be reduced. For example, the computing device (e.g., the control computing device 144, 344) may reduce the first quantity of torque provided by the motorized assistance during the second step of the walking motion for the paretic leg. The first quantity of torque may be reduced based on the movement satisfying the target configuration. For example, the computing device may decrease the amount of the torque to a level below the first quantity of torque. Alternatively or additionally, the computing device may decrease the amount of time motorized assistance is provided at the first quantity of torque. The first quantity of electrical stimuli may be provided to the portion of the paretic leg during the second step of the walking motion for the paretic leg. The first quantity of electrical stimuli may be simultaneously provided to the portion of the paretic leg with the reduced first quantity of torque provided by the motorized assistance during the second step of the walking motion.

Second data associated with the paretic leg during the second step of the walking motion may be received. For example, the computing device (e.g., the control computing device 144, 344) may receive the second data associated with the paretic leg during the second step of the walking motion. The second data associated with the paretic leg during the second step of the walking motion may be received from one or more sensors (e.g., the one or more sensors 129, 134-142, 154, 156). The second data associated with the paretic leg during the second step of the walking motion may include, but are not limited to, acceleration data, velocity data, angular velocity data, orientation data, muscle activity data, and position data. The second data associated with the paretic leg during the second step of the walking motion may be received via wired or wireless communication such as Wi-Fi, Bluetooth, Bluetooth Low Energy (BLE), Zigbee, or any other known wired/wireless protocol with the one or sensors.

A deviation of the paretic leg from the target configuration for the paretic leg during the second step of the walking motion may be determined. For example, the computing device may determine the deviation of the paretic leg from the target configuration of the paretic leg based on the second data. The deviation of the paretic leg may comprise a difference between a target angular configuration for the paretic leg during the second step and an actual angular configuration for the paretic leg during the second step. The deviation of the paretic leg during the second step may be determined based on a plurality of weighting factors. The plurality of weighting factors may comprise a weighting factor minimizing the provision of motorized assistance and a weighting factor maximizing muscle contribution of the paretic leg by electrical stimuli.

One or more of the first quantity of torque provided by the motorized assistance or the first quantity of electrical stimuli provided to the portion of the paretic leg during a next step of the walking motion for the paretic leg may be modified. For example, the computing device may modify, based on the deviation, one or more of the first quantity of torque provided by the motorized assistance or the first quantity of electrical stimuli provided to the portion of the paretic leg during the next step of the walking motion for the paretic leg. For example, the computing device may increase an amount of torque to a level above the first quantity of torque or decrease the amount of the torque to a second level below the first quantity of torque. The computing device may also increase an amount of the electrical stimuli to a level above the first quantity of electrical stimuli or decrease the amount of the electrical stimuli to a second level below the first quantity of electrical stimuli. One or more of the modified quantity of torque or the modified quantity of electrical stimuli may be provided to the portion of the paretic leg during the next step of the walking motion for the paretic leg.

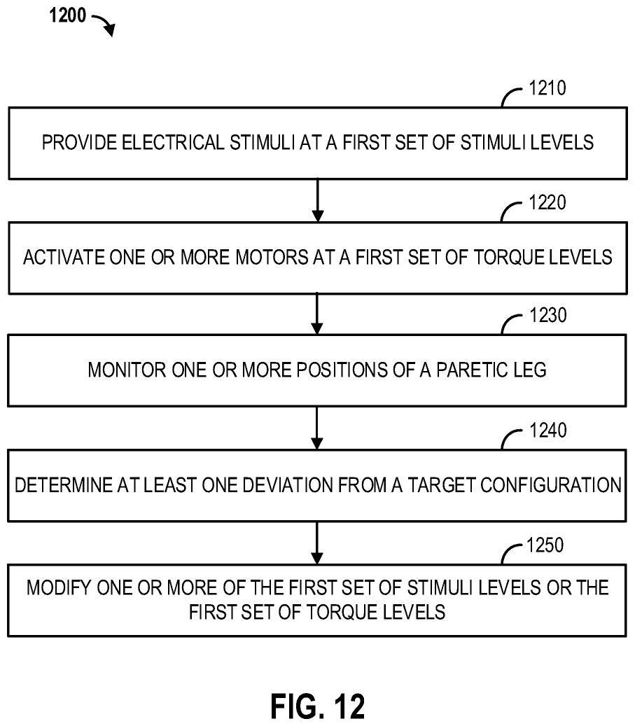

FIG. 12 shows an example method 1200 for providing electrical and/or mechanical assistance for leg movement. The method 1200 may be performed by any device such as the control computing device 144, 344, the user device 148, and a computing device in communication with the one or more sensors 129, 134-142, 154, 156, the one or more motors 128, 152, and/or the one or more electrodes 130A-B, 132. Although the method 1200 is described for a paretic leg (e.g., the paretic leg 104), the method 1200 may also apply to the other paretic leg when both legs of a user (e.g., the user 102) are paretic.

At step 1210, electrical stimuli may be provided to one or more portions of a paretic leg at a first set of stimuli levels during one or more phases of a first step of a walking motion. For example, a computing device (e.g., the control computing device 144, 344) may provide the electrical stimuli to one or more portions of the paretic leg at the first set of stimuli levels during the first step of the walking motion. The one or more portions of the paretic leg may indicate one or more weak or inactive muscles in the paretic leg. The electrical stimuli at the first set of stimuli levels may recruit the one or more weak or inactive muscles to force the first step of the walking motion.

At step 1220, one or more motors of an exoskeleton for the paretic leg may be activated at a first set of torque levels during the one or more phases of the first step of the walking motion. For example, the computing device may activate the one or more motors of the exoskeleton at the first set of torque levels during the one or more phases of the first step of the walking motion. The first set of stimuli levels and/or the first set of torque levels may be determined based on the strength and availability of one or more weak or inactive muscles in the paretic leg. Providing the electrical stimuli at the first set of stimuli levels and activating the one or more motors at the first set of torque levels may be performed simultaneously.

At step 1230, one or more positions of the paretic leg during the first step of the walking motion may be monitored. For example, the computing device may monitor the one or more positions of the paretic leg during the first step of the walking motion based on one or more sensors (e.g., the one or more sensors 129, 134-142, 154, 156). The one or more sensors may comprise a thigh sensor (e.g., the thigh sensor 134), a shank sensor (e.g., the shank sensor 136), a heel-strike sensor (e.g., the heel-strike sensor 138), a foot sensor (e.g., the foot sensor 140), and a hip sensor. The computing device may receive, from the one or more sensors, data associated with the paretic leg during the first step of the walking motion. The sensor data may include, but are not limited to, acceleration data, velocity data, angular velocity data, orientation data, muscle activity data, and position data.

At step 1240, a deviation of the paretic leg from a target angular configuration may be determined. For example, the computing device may determine the deviation of the paretic leg from the target angular configuration during the first step of the walking motion based on the one or more positions of the paretic leg during the first step of the walking motion. The one or more positions of the paretic leg may indicate a terminal configuration to accept weight at the end of the first step of the walking motion. The deviation of the paretic leg may indicate a terminal error between the target angular configuration and an actual angular configuration that is determined based on the one or more positions of the paretic leg at the end of the first step. The target angular configuration and/or the actual angular configuration may comprise a hip flexion angle, a knee flexion angle, and a knee extension angle. For example, the hip flexion angle of the target angular configuration may be 30 degrees. The knee flexion angle of the target angular configuration may be 50 degrees. The knee extension angle of the target angular configuration may be 0 degree. The deviation of the paretic leg may be determined based on a plurality of weighting factors. The plurality of weighting factors may comprise a first weighting factor minimizing motorized assistance from the one or more motors and a second weight factor maximizing muscle contribution by the electrical stimuli.

At step 1250, one or more of the first set of stimuli levels or the first set of torque levels may be modified for the next step of the walking motion. For example, the computing device may modify one or more of the first set of stimuli levels or the first set of torque levels based on the deviation of the paretic leg from the target angular configuration. For example, the computing device may determine a plurality of scaling factors associated with hip flexion, knee flexion, and knee extension based on the deviation. The computing device may apply the plurality of scaling factors to the first set of stimuli levels. The computing device may then generate a second set of stimuli levels for the next step of the walking motion as the modification of the first set of stimuli levels. The computing device may determine a plurality of torque impulses associated with the hip flexion, knee flexion and knee extension based on the deviation. The computing device may apply the plurality of torque impulses to the first set of torque levels. The computing device may then generate a second set of torque levels for the next step of the walking motion as the modification of the first set of torque levels. The computing device may provide second electrical stimuli to the one or more portions of the paretic let at the second set of stimuli levels during one or more phases of the next step of the walking motion. The computing device may activate the one or more motors of the exoskeleton at the second set of torque levels during the one or more phases of the next step of the walking motion. The second electrical stimuli provided at the second set of stimuli levels and motor assistance activated at the second set of torque levels may be provided/performed simultaneously during the one or more phases of the next step of the walking motion.

It is noted that the methods 900-1200 described in FIGS. 9-12 may be performed using various Artificial Intelligence or machine-learning techniques to control electrical and/or mechanical assistance for leg movement. Examples of machine-learning techniques for adaptive control of electrical and/or mechanical assistance may include, but are not limited to, iterative learning control (ILC), neural networks, reinforcement learning, fuzzy logic systems, Kalman filtering, and ensemble learning. For example, neural network techniques such as function approximation and/or adaptive neural networks may be used to control electrical stimulation and/or motor assistance based on the system dynamics and system performance parameters for the systems 100-350 shown in FIGS. 1-3B.

Reinforcement learning techniques such as model-free learning and/or policy gradient methods may be used to control electrical stimulation and/or motor assistance based on the system optimization for the systems 100-350 shown in FIGS. 1-3B. The fuzzy logic systems such as adaptive fuzzy control may be used to control electrical stimulation and/or motor assistance based on handling uncertainties and imprecise information of the system dynamics for the systems 100-350 shown in FIGS. 1-3B. The Kalman filtering technique such as adaptive Kalman filtering may be used to control electrical stimulation and/or motor assistance based on the system state and parameter in real-time for the systems 100-350 shown in FIGS. 1-3B. The ensemble learning technique such as combining models may be used to control electrical stimulation and/or motor assistance based on multiple model combination for the systems 100-350 shown in FIGS. 1-3B.

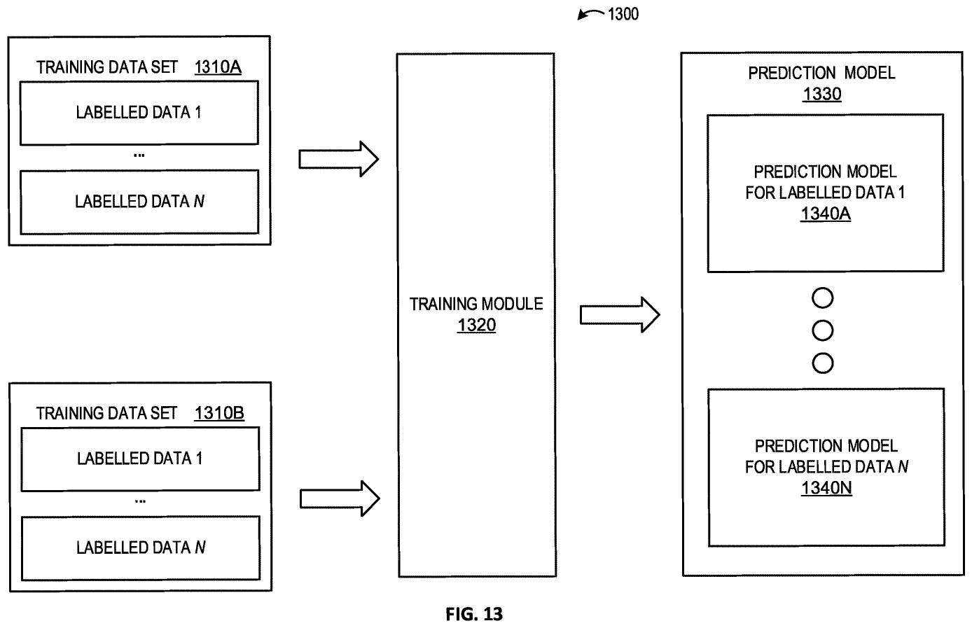

FIG. 13 shows an example system 1300 for machine-learning model training. The system 1300 may be configured to use machine-learning techniques to train, based on an analysis of a plurality of training datasets 1310A-1310B by a training module 1320, a prediction model 1330. Functions of the system 1300 described herein may be performed, for example, by the control computing device 144, the user device 148 and/or another computing device in communication with the sensors 129, 134-142, 154, 156, the motors 128, 152, and/or the electrodes 130A-B, 132 via wired or wireless communication. The plurality of training datasets 1310A-1310B may be associated with input data or annotated data described herein. For example, the training dataset 1310A may comprise one or more labelled data (e.g., labelled data 1-N). Each of the one more labelled data in the training dataset 1310A may comprise one or more inputs (e.g., input features) and corresponding known outputs associated with the one or more inputs. For example, labelled data 1 of the training dataset 1310A may comprise an amount of rotation, acceleration, velocity, angular velocity, orientation, position, and/or muscle activity received from the sensors 129, 134-142, 154, 156.

The training datasets 1310A, 1310B may be based on, or comprise, the data stored in database of the control computing device 144, the user device 144, and/or another computing device in communication with the sensors 129, 134-142, 154, 156, the motors 128, 152, and/or the electrodes 130A-B, 132. Such data may be randomly assigned to the training dataset 1310A, the training dataset 1310B, and/or to a testing dataset. In some implementations, assignment may not be completely random and one or more criteria or methods may be used during the assignment. For example, the training dataset 1310A and/or the training dataset 1310B may be generated based on one or more positions of the paretic leg(s) during the step(s) of walking motion. The one or more positions of the paretic leg(s) may be monitored by the one or more sensors 129, 134-142, 154, 156. In general, any suitable method may be used to assign the data to the training and/or testing datasets.

The training module 1320 may train the prediction model 1330 by determining/extracting the features from the training dataset 1310A and/or the training dataset 1310B in a variety of ways. For example, the training module 1320 may determine/extract a feature set from the training dataset 1310A and/or the training dataset 1310B to estimate a deviation of the paretic leg from the target configuration described in FIGS. 9-12. The training module 1320 may determine/extract a feature set from the training dataset 1310A and/or the training dataset 1310B to determine the quantity of electrical stimuli and/or the quantity of torque described in FIGS. 9-12. The training module 1320 may use the feature sets to generate prediction models 1340A-1340N for the prediction of the quantity of electrical stimuli and/or the quantity of torque.

The training dataset 1310A and/or the training dataset 1310B may be analyzed to determine any dependencies, associations, and/or correlations between features in the training dataset 1310A and/or the training dataset 1310B. The identified correlations may have the form of a list of features that are associated with different labeled predictions. The term “feature,” as used herein, may refer to any characteristic of an item of data that may be used to determine whether the item of data falls within one or more specific categories or within a range. A feature selection technique may comprise one or more feature selection rules. The one or more feature selection rules may comprise a feature occurrence rule. The feature occurrence rule may comprise determining which features in the training dataset 1310A occur over a threshold number of times and identifying those features that satisfy the threshold as candidate features. For example, any features that appear greater than or equal to 5 times in the training dataset 1310A may be considered as candidate features. Any features appearing less than 5 times may be excluded from consideration as a feature. Other threshold numbers may be used as well.

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training dataset 1310A to generate a first list of features. A final list of candidate features may be analyzed according to additional feature selection techniques to determine one or more candidate feature groups (e.g., groups of features that may be used to determine a prediction). Any suitable computational technique may be used to identify the candidate feature groups using any feature selection technique such as filter, wrapper, and/or embedded methods. One or more candidate feature groups may be selected according to classifiers and/or a statistical method. The classifiers and/or statistical method may include, for example, Pearson's correlation, linear discriminant analysis, analysis of variance (ANOVA), chi-square, combinations thereof, and the like. The selection of features according to filter methods are independent of any machine-learning algorithms used by the system 1300. Instead, features may be selected on the basis of scores in various statistical tests for their correlation with the outcome variable (e.g., a prediction).

As another example, one or more candidate feature groups may be selected according to a wrapper method. A wrapper method may be configured to use a subset of features and train the prediction model 1330 using the subset of features. Based on the inferences that may be drawn from a previous model, features may be added and/or deleted from the subset. Wrapper methods include, for example, forward feature selection, backward feature elimination, recursive feature elimination, combinations thereof, and the like. For example, forward feature selection may be used to identify one or more candidate feature groups. Forward feature selection is an iterative method that begins with no features. In each iteration, the feature which best improves the model is added until an addition of a new variable does not improve the performance of the model. As another example, backward elimination may be used to identify one or more candidate feature groups. Backward elimination is an iterative method that begins with all features in the model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to the absolute value of the magnitude of coefficients and ridge regression performs L2 regularization which adds a penalty equivalent to the square of the magnitude of coefficients.

After the training module 1320 has generated a feature set(s), the training module 1320 may generate the prediction models 1340A-1340N based on the feature set(s). A machine-learning-based prediction model (e.g., any of the prediction models 1340A-1340N) may refer to a complex mathematical model for the prediction of electrical/mechanical assistance for one or both paretic legs. The complex mathematical model for the prediction of optimum electrical stimulation and/or motor assistance may be generated using machine-learning techniques as described herein. For example, a machine-learning-based iterative learning control model may determine the predicted quantity of electrical stimuli/torque during a next step of the walking motion for one or both paretic legs. The training module 1320 may use the feature sets extracted from the training dataset 1310A and/or the training dataset 1310B to build the prediction models 1340A-1340N for the adaptive control of the electrical/mechanical assistance. In some examples, the prediction models 1340A-1340N may be combined into a single prediction model 1340 (e.g., an ensemble model). Similarly, the prediction model 1330 may represent a single model containing a single or a plurality of prediction models 1340 and/or multiple models containing a single or a plurality of prediction models 1340 (e.g., an ensemble model). It is noted that the training module 1320 may be part of the control computing device 144, the user device 148, and/or another computing device in communication with the sensors 129, 134-142, 154, 156, the motors 128, 152, and/or the electrodes 130A-B, 132.

The extracted features (e.g., one or more candidate features) may be combined in the prediction models 1340A-1340N that are trained using a machine-learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting classification model 1330 may comprise a decision rule or a mapping for each candidate feature in order to assign a prediction to a class.

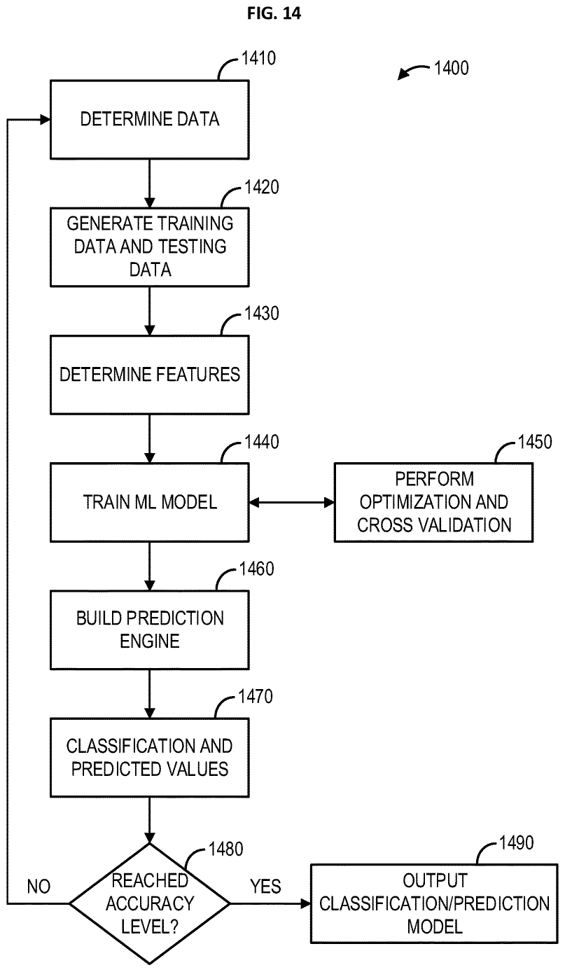

FIG. 14 is a flowchart illustrating an example training method 1400 for generating the prediction model 1330 using the training module 1320. The training module 1320 may implement supervised, unsupervised, and/or semi-supervised (e.g., reinforcement based) learning. The method 1400 illustrated in FIG. 14 is an example of a supervised learning method; variations of this example of training method may be analogously implemented to train unsupervised and/or semi-supervised machine-learning models. The method 1400 may be implemented by any of the devices shown in any of the systems 100-350 in FIGS. 1-3B. For example, the method 1400 may be performed by the control computing device 144, the user device 148 and/or another computing device in communication with the sensors 129, 134-142, 154, 156, the motors 128, 152, and/or the electrodes 130A-B, 132 via wired or wireless communication.

At step 1410, the training method 1400 may determine (e.g., access, receive, retrieve, etc.) first training data and second training data (e.g., the training datasets 1310A-1310B). The first training data and the second training data may each comprise one or more labelled data. The one more labelled data may comprise one or more inputs (e.g., input features) and corresponding known outputs associated with the one or more inputs. For example, one or more labelled data of the training data may comprise an amount of rotation, acceleration, velocity, angular velocity, orientation, position, and/or muscle activity received from the sensors 129, 134-142, 154, 156. The training method 1400 may generate, at step 1420, a training dataset and a testing dataset. The training dataset and the testing dataset may be generated by randomly assigning data from the first training data and/or the second training data to either the training dataset or the testing dataset. In some implementations, the assignment of data as training or test data may not be completely random. For example, the training dataset and/or the testing dataset may be generated based on one or more positions of paretic leg(s) during the step(s) of walking motion. The one or more positions of the paretic leg(s) may be monitored by the one or more sensors 129, 134-142, 154, 156.

The training method 1400 may determine (e.g., extract, select, etc.), at step 1430, one or more features that may be used to, for example, estimate a deviation of the paretic leg from the target configuration described in FIGS. 9-12. The one or more features may comprise a set of features. As an example, the training method 1400 may determine a set of features from the first training data. As another example, the training method 1400 may determine a set of features from the second training data.

The training method 1400 may train one or more machine-learning models (e.g., one or more classification models, one or more prediction models, neural networks, deep-learning models, etc.) using the one or more features at step 1440. In one example, the machine-learning models may be trained using supervised learning. In another example, other machine-learning techniques may be used, including unsupervised learning and semi-supervised. The machine-learning models trained at step 1440 may be selected based on different criteria depending on the problem to be solved and/or data available in the training dataset. For example, machine-learning models may suffer from different degrees of bias. Accordingly, more than one machine-learning model may be trained at 1440, and then optimized, improved, and cross-validated at step 1450.

The training method 1400 may select one or more machine-learning models to build the prediction model 1330 at step 1460. The classification/prediction model 1330 may be evaluated using the testing dataset. The prediction model 1330 may analyze the testing dataset and generate predicted values (e.g., the quantity of electrical stimuli, the quantity of torque) at step 1470. Classification and/or prediction values may be evaluated at step 1480 to determine whether such values have achieved a desired accuracy level. Performance of the prediction model 1330 may be evaluated in a number of ways based on a number of true positives, false positives, true negatives, and/or false negatives classifications of the plurality of data points indicated by the prediction model 1330. Generally, recall refers to a ratio of true positives to a sum of true positives and false negatives, which quantifies a sensitivity of the classification/prediction model 1330. Similarly, precision refers to a ratio of true positives a sum of true and false positives. When such a desired accuracy level is reached, the training phase ends and the classification/prediction model 1330 may be output at step 1490; when the desired accuracy level is not reached, however, then a subsequent iteration of the training method 1400 may be performed starting at step 1410 with variations such as, for example, considering a larger collection of labelled data from speech corpus and non-speech corpus. The prediction model 1330 may be output at step 1490.

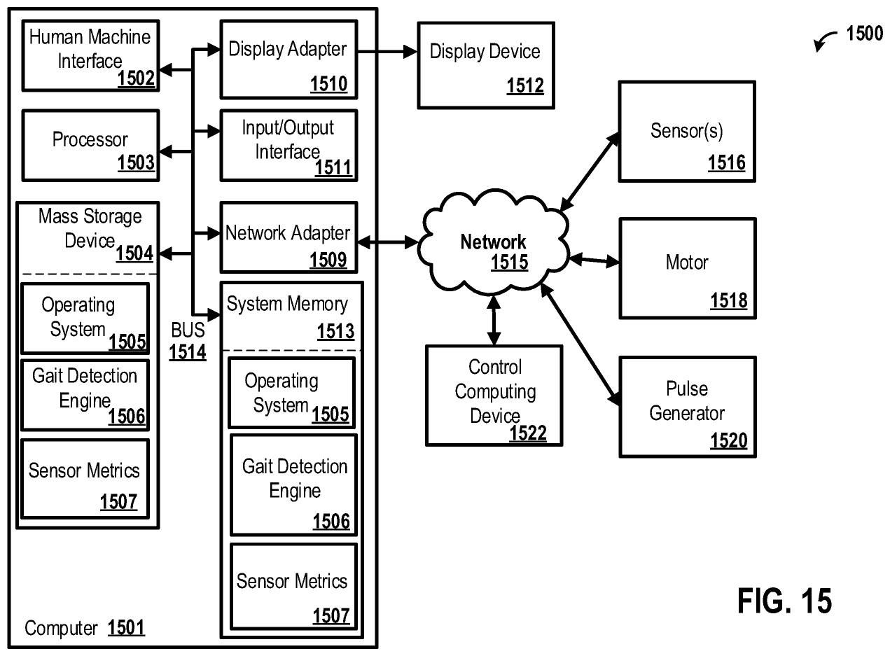

FIG. 15 shows a system 1500 for providing electrical and/or mechanical assistance for leg movement. The user device 148, the control computing device 144, or another computing device may be a computer 1501 as shown in FIG. 15.

The computer 1501 may comprise one or more processors 1503, a system memory 1513, and a bus 1514 that couples various components of the computer 1501 including the one or more processors 1503 to the system memory 1513. In the case of multiple processors 1503, the computer 1501 may utilize parallel computing.

The bus 1514 may comprise one or more of several possible types of bus structures, such as a memory bus, memory controller, a peripheral bus, an accelerated graphics port, and a processor or local bus using any of a variety of bus architectures.

The computer 1501 may operate on and/or comprise a variety of computer-readable media (e.g., non-transitory). Computer-readable media may be any available media that is accessible by the computer 1501 and includes, non-transitory, volatile and/or non-volatile media, and removable and non-removable media. The system memory 1513 has computer-readable media in the form of volatile memory, such as random access memory (RAM), and/or non-volatile memory, such as read-only memory (ROM). The system memory 1513 may store data and/or program modules such as an operating system 1505, the gait detection engine 1506, and sensor metrics 1507 that are accessible to and/or are operated on by the one or more processors 1503.

The computer 1501 may also comprise other removable/non-removable, volatile/non-volatile computer storage media. The mass storage device 1504 may provide non-volatile storage of computer code, computer-readable instructions, data structures, program modules, and other data for the computer 1501. The mass storage device 1504 may be a hard disk, a removable magnetic disk, a removable optical disk, magnetic cassettes or other magnetic storage devices, flash memory cards, CD-ROM, digital versatile disks (DVD) or other optical storage, RAM, ROM, electrically erasable programmable read-only memory (EEPROM), and the like.

Any number of program modules may be stored on the mass storage device 1504. An operating system 1505, the gait detection engine 1506, and sensor metrics 1507 may be stored on the mass storage device 1504. One or more of the operating system 1505, gait detection engine 1506, and sensor metrics 1507 (or some combination thereof) may comprise one or more program modules.

A user 102 may enter commands and information into the computer 1501 via an input device. Such input devices may include, but are not limited to, a keyboard, pointing device (e.g., a computer mouse or remote control), a microphone, a joystick, a scanner, tactile input devices such as gloves, and other body coverings, a motion sensor, and the like These and other input devices may be connected to the one or more processors 1503 via a human-machine interface 1502 that is coupled to the bus 1514, but may be connected by other interface and bus structures, such as a parallel port, game port, an IEEE 1394 Port (also known as a Firewire port), a serial port, network adapter 1509, and/or a universal serial bus (USB).

A display device 1512 may also be connected to the bus 1514 via an interface, such as a display adapter 1510. It is contemplated that the computer 1501 may have zero displays or more than one display adapter 1510 and the computer 1501 may have more than one display device 1512. A display device 1512 may be a monitor, an LCD (Liquid Crystal Display), a light-emitting diode (LED) display, a television, smart lens, smart glass, and/or a projector. In addition to the display device 1512, other output peripheral devices may comprise components such as speakers (not shown) and a printer (not shown) which may be connected to the computer 1501 via Input/Output Interface 1511. Any step and/or result of the methods may be output (or caused to be output) in any form to an output device. Such output may be any form of visual representation, including, but not limited to, textual, graphical, animation, audio, tactile, and the like. The display 1512 and computer 1501 may be part of one device, or separate devices.

The computer 1501 may operate in a networked environment using logical connections to one or more other devices, such as the one or more sensors 1516, one or more motors 1518, and/or a pulse generator 1520. The one or more sensors 1516 may comprise the sensors 129, 134-142, 154, 156 of FIG. 1. The one or more motors 1518 may comprise one or both of the motors 128, 152, and the pulse generator 1520 may comprise the pulse generator 202 of FIG. 2. Logical connections between the computer 1501, the one or more sensors 1516, the one or more motors 1518, and the pulse generator 1520 may be made via a network 1515, such as a local area network (LAN) and/or a general wide area network (WAN) and one or more network devices (e.g., a router, an edge device, an access point or other common network nodes, such as a gateway). Such network connections may be through a network adapter 1509. The network adapter 1509 may be implemented in both wired and wireless environments. Such networking environments are conventional and commonplace in dwellings, offices, enterprise-wide computer networks, intranets, and the Internet.

Application programs and other executable program components such as the operating system 1505, the gait detection engine 1506, and the sensor metrics 1507 are shown herein as discrete blocks, although it is recognized that such programs and components may reside at various times in different storage components of the computing device 1501, and are executed by the one or more processors 1503 of the computer 1501. Any of the disclosed methods may be performed by processor-executable instructions embodied on computer-readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
providing a first quantity of electrical stimuli to a portion of a paretic leg during a first step of a walking motion;
providing motorized assistance for the paretic leg at a first quantity of torque during the first step of the walking motion;
receiving data associated with the paretic leg during the first step of the walking motion;
determining, based on the data, a deviation of the paretic leg, during the first step of the walking motion, from a target configuration for the paretic leg during the first step of the walking motion; and
modifying, based on the deviation, the first quantity of electrical stimuli during a next step of the walking motion for the paretic leg.

2. The method of claim 1, wherein providing the first quantity of electrical stimuli causes a contraction of a muscle in the portion of the paretic leg.

3. The method of claim 1, wherein determining the deviation of the paretic leg, during the first step of the walking motion, from a target configuration for the paretic leg comprises:
determining a first position of the portion of the paretic leg during the first step at a first time;
determining a target position for the portion of the paretic leg during the first step at the first time; and
determining the first position of the portion of the paretic leg varies from the target position for the portion of the paretic leg at the first time.

4. The method of claim 1, wherein determining the deviation of the paretic leg, during the first step of the walking motion, from a target configuration for the paretic leg comprises:
determining a first angular velocity of the portion of the paretic leg during the first step at a first time;
determining a target angular velocity for the portion of the paretic leg during the first step at the first time; and
determining a difference between the first angular velocity of the portion of the paretic leg and the target angular velocity for the portion of the paretic leg at the first time satisfies a threshold.

5. The method of claim 1, wherein modifying the first quantity of electrical stimuli comprises one or more of increasing an amount of the electrical stimuli to a level above the first quantity of electrical stimuli, decreasing the amount of the electrical stimuli to a second level below the first quantity of electrical stimuli, increasing an amount of time the electrical stimuli is provided to the portion of the paretic leg, decreasing the amount of time the electrical stimuli is provided to the portion of the paretic leg, increasing a frequency the electrical stimuli is provided to the portion of the paretic leg, or decreasing the frequency the electrical stimuli is provided to the paretic leg.

6. The method of claim 1, wherein providing motorized assistance comprises activating at least one motor of an exoskeleton coupled to the paretic leg.

7. The method of claim 1, further comprising modifying, based on the deviation, the first quantity of torque provided by the motorized assistance during the next step of the walking motion for the paretic leg.

8. The method of claim 1, wherein providing the electrical stimuli and at least a portion of providing the motorized assistance are performed simultaneously.

9. The method of claim 1, wherein the deviation of the paretic leg comprises a difference between a target angular configuration for the paretic leg and an actual angular configuration for the paretic leg during the first step.

10. The method of claim 1, wherein the deviation of the paretic leg is determined based on a plurality of weighting factors, wherein the plurality of weighting factors comprise a first weighting factor minimizing the provision of motorized assistance and a second weighting factor maximizing muscle contribution of the paretic leg by electrical stimuli.

11. The method of claim 1, further comprising providing the modified quantity of electrical stimuli to the portion of the paretic leg during one or more phases of the next step of the walking motion.

12. A method comprising:
providing a first quantity of electrical stimuli to a portion of a paretic leg during a first step of a walking motion;
providing motorized assistance for the paretic leg at a first quantity of torque during the first step of the walking motion;
receiving data associated with the paretic leg during the first step of the walking motion;
determining, based on the data, a deviation of the paretic leg, during the first step of the walking motion, from a target configuration for the paretic leg during the first step of the walking motion; and
modifying, based on the deviation, the first quantity of torque provided by the motorized assistance during a next step of the walking motion for the paretic leg.

13. The method of claim 12, further comprising modifying, based on the deviation, the first quantity of electrical stimuli during a next step of the walking motion for the paretic leg.

14. The method of claim 12, wherein modifying the first quantity of torque comprises one or more of increasing an amount of torque to a level above the first quantity of torque, decreasing the amount of the torque to a second level below the first quantity of torque, increasing an amount of time motorized assistance is provided at the first quantity of torque, or decreasing the amount of time motorized assistance is provided at the first quantity of torque.

15. The method of claim 12, wherein determining the deviation of the paretic leg, during the first step of the walking motion, from a target configuration for the paretic leg comprises:
determining a first position of the portion of the paretic leg during the first step at a first time;
determining a target position for the portion of the paretic leg during the first step at the first time; and
determining the first position of the portion of the paretic leg varies from the target position for the portion of the paretic leg at the first time.

16. The method of claim 12, wherein determining the deviation of the paretic leg, during the first step of the walking motion, from a target configuration for the paretic leg comprises:
determining a first angular velocity of the portion of the paretic leg during the first step at a first time;
determining a target angular velocity for the portion of the paretic leg during the first step at the first time; and
determining a difference between the first angular velocity of the portion of the paretic leg varies and the target angular velocity for the portion of the paretic leg at the first time satisfies a threshold.

17. The method of claim 1, wherein providing the electrical stimuli and at least a portion of providing the motorized assistance are performed simultaneously, and wherein providing the motorized assistance comprises activating at least one motor of an exoskeleton coupled to the paretic leg.

18. A method comprising:
providing a first quantity of electrical stimuli to a portion of a paretic leg during a first step of a walking motion;
providing motorized assistance for the paretic leg at a first quantity of torque during the first step of the walking motion;
receiving data associated with the paretic leg during the first step of the walking motion;
determining, based on the data, a movement of the paretic leg, during the first step of the walking motion, satisfies a target configuration for the paretic leg during the first step of the walking motion; and
reducing, based on the movement satisfying the target configuration, the first quantity of torque provided by the motorized assistance during a second step of the walking motion for the paretic leg.

19. The method of claim 18, further comprising providing the first quantity of electrical stimuli to the portion of the paretic leg during the second step of the walking motion for the paretic leg.

20. The method of claim 18, further comprising:
receiving second data associated with the paretic leg during the second step of the walking motion;
determining, based on the second data, a deviation of the paretic leg, during the second step of the walking motion, from the target configuration for the paretic leg during the second step of the walking motion; and
modifying, based on the deviation, one or more of the first quantity of torque provided by the motorized assistance or the first quantity of electrical stimuli provided to the portion of the paretic leg during a next step of the walking motion for the paretic leg.
